(19) United States
(12) Patent Application Publication
(10) Pub. No.: US 2024/0174755 A1
(43) Pub. Date: May 30, 2024

(54) PLATELET DERIVED GROWTH FACTOR RECEPTOR (PDGFR) ANTIBODIES, CONJUGATES, COMPOSITIONS, AND USES THEREOF

(71) Applicant: BIORION TECHNOLOGIES B.V., GX Groningen (NL)

(72) Inventors: Hermannus STEEN, GX Groningen (NL); Gustaaf Johan Marie VAN SCHARRENBURG, GX Groningen (NL)

(73) Assignee: BIORION TECHNOLOGIES B.V., GX Groningen (NL)

(21) Appl. No.: 17/760,252

(22) PCT Filed: Feb. 5, 2021
(86) PCT No.: PCT/NL2021/050074

§ 371 Date: Aug. 5, 2022

(30) Foreign Application Priority Data
Feb. 7, 2020 (EP) 20156203.0

(51) Int. Cl. C07K 16/28; A61K 45/06; A61K 47/68; A61K 49/00; A61K 51/10

(52) U.S. Cl. CPC C07K 16/2863; A61K 45/06; A61K 47/6849; A61K 49/0002; A61K 51/103; C07K 2317/31; C07K 2317/35; C07K 2317/569

(57) ABSTRACT

The invention relates to antibodies against the Platelet Derived Growth Factor Receptor beta (PDGFR β), and the use thereof in diagnostic and/or therapeutic applications. In particular, it provides a (VHH) antibody that specifically binds PDGFR β with an apparent binding affinity of less than 10 nM, preferably less than 5 nM, and which does not activate PDGFR β. Also provided are PDGFR β antibodies and conjugates thereof, and their application in the targeted delivery of a diagnostic agent, a therapeutic agent or a combination thereof to a tissue in a subject, in particular to fibrotic tissue comprising activated myofibroblasts.

### CROSS-REFERENCE TO RELATED APPLICATION

This application is the U.S. national phase of PCT Application No. PCT/NL2021/050074 filed on Feb. 5, 2021, which claims priority to EP Patent Application No. 20156203.0 filed on Feb. 7, 2020, the disclosures of which are incorporated in their entirety by reference herein.

### SEQUENCE LISTING

The text file BiOrion_st25 of size 55 KB created Jul. 26, 2022, filed herewith, is hereby incorporated by reference.

The invention relates to antibodies against the Platelet Derived Growth Factor Receptor beta (PDGFR β), and the use thereof in diagnostic and/or therapeutic applications. Among others, it relates to PDGFR β targeting antibodies and conjugates thereof, and their application in the targeted delivery of a diagnostic agent, a therapeutic agent or a combination thereof to a tissue in a subject, in particular to fibrotic tissue comprising activated myofibroblasts.

Platelet-derived growth factor (PDGF) family has 4 types: PDGF-A, -B, -C, and -D), and two receptors: PDGFR α and β. These receptors have differential binding specificity for the various PDGF dimers. PDGFR α ligates PDGF A, B, and C chains, and PDGFR β binds PDGF B and D chains. The two PDGF receptors are structurally and functionally related, and PDGF binding results in receptor dimerization and the formation of

PDGFR αα, ββ, and αβ receptor dimers. For receptor activation, PDGF AA and PDGF CC require PDGFR αα, or αβ dimers, PDGF DD requires PDGFR ββ, or αβ dimers, whereas PDGF AB and PDGF BB can activate PDGFR αα, αβ, or ββ dimers. PDGF binding to its receptor results in receptor dimerization, and activation of tyrosine kinase activity, leading to activation of protein kinase C, and intracellular calcium signaling pathways.

It has been shown that the cause of liver fibrosis, bone marrow fibrosis, lung fibrosis, and kidney fibrosis is related to the overexpression of PDGF family and PDGFR. Three steps in chronic liver diseases are 1) hepatitis, 2) liver fibrosis, and 3) liver cirrhosis. Chronic fibrosis disrupts the essential structure of liver sinusoids, impairing the function of the liver and eventually leading to cirrhosis. If liver fibrosis cannot be treated, it will eventually lead to liver cirrhosis. It has been shown that liver fibrosis is caused by activation of hepatic stellate cells (HSC) that transdifferentiate into myofibroblasts in liver tissue. Overexpression of PDGF-C and PDGF receptors at mRNA and protein levels is one of the earliest events. Activated HSC and myofibroblasts produce a number of profibrotic cytokines and growth factors that perpetuate the fibrotic process through paracrine and autocrine effects. PDGF-BB and TGF-β1 are two key factors in fibrogenesis. Liver fibrosis development leads to liver tissue hyperemia and liver steatosis formation, and eventually liver cancer. Thus, any medication which can prevent or treat liver fibrosis is a good adjunct therapy for liver cancer.

Besides their involvement in organ fibrosis, it has recently been highlighted that fibroblasts and myofibroblasts also have a pivotal role in tumor progression, invasion and metastasis. As reviewed by Yazdani et al. (Adv. Drug Delivery Rev 121 (2017) 101-116), myofibroblast targeting has gained tremendous attention in order to inhibit the progression of incurable fibrotic diseases, or to limit the myofibroblast-induced tumor progression and metastasis. It is generally accepted that myofibroblasts are key players in fibrosis progression in three major organs: liver, kidneys and lungs as well as in cancer. Accordingly, targeting technologies to myofibroblasts in the context of the above-mentioned organs and tumor microenvironment are receiving a great interest, in particular to design new strategies to develop novel diagnostics and therapeutics against fibrosis and cancer.

The therapeutic targeting strategies to inhibit myofibroblast function can be categorized into (i) small molecule drugs/inhibitors e.g. receptor tyrosine kinases inhibitors such as RhoA kinase, ERK, JNK etc.; signaling pathways inhibitors such as TGF-β, PDGFR β, Hedgehog, Notch, Wnt, endothelin-1, and siRNA. (ii) Monoclonal antibodies that can identify and bind to the targets on the cell surface or outside the cells. (iii) Targeted delivery systems consisting of the targeting moiety to a delivery vehicle or protein carrying a therapeutic agent conjugated to targeting ligands.

The expression of PDGF receptors on myofibroblasts has been shown to be tissue specific in different fibrotic diseases. For example, PDGFR α expression on lung myofibroblasts can be induced or suppressed by different stimuli in pulmonary diseases, while they express PDGFR β constitutively. In contrast, PDGFR β expression on liver myofibroblasts is extremely inducible and upregulated during liver injury, and is a hallmark of early HSCs activation (Bonner, Cytokine Growth Factor Rev., 15 (2004), pp. 255-273). PDGF expression has also been correlated with myofibroblasts differentiation and proliferation, and subsequent ECM deposition, both in experimental models and human diseases. PDGF antagonism and pharmacological inhibition of PDGFR β has shown to be a promising therapeutic approach and is therefore a potential target in organ fibrosis as well as in tumor growth and metastasis. The myofibroblasts' PDGF receptors have been effectively utilized for targeted delivery of compounds to treat organ fibrosis or tumor growth (Bansal et al., PLOS One, 9 (2014), Article e89878; Poosti et al., FASEB J., 29 (2015), pp. 1029-1042; Prakash et al., J. Control. Release, 148 (2010), Article e116; Prakash et al., J. Control. Release, 145 (2010), pp. 91-101; van Dijk et al., Front. Med. (Lausanne), 2 (2015), p. 72). US2011/0282033 relates to amino acid sequences that are directed against receptors for growth factors, compounds comprising such sequences, as well as nucleic acid sequences encoding the same. In one embodiment the amino acid sequences are Nanobodies™M, including nanobodies against the PDGF receptor.

Several promising therapeutic antibodies and aptamers for targeting the PDGF receptors in liver fibrosis which are currently in advanced preclinical studies or clinical trials (Borkham-Kamphorst et al., Cytokine Growth Factor Rev., 28 (2016), pp. 53-61).

Recognizing the potential of targeting PDGF receptors as clinically feasible therapeutic and diagnostic approach in fibrosis and cancer, the present inventors aimed at providing targeting means that show a high affinity for the PDGFR β. In particular, they sought to identify antibodies that specifically bind to PDGFR β, as expressed e.g. on activated myofibroblasts, with high affinity. Preferably, the antibodies show a binding affinity for (dimeric) PDGFR β of less than 10 nM, more preferably of about 1 nM or less. Also, the targeting antibody binding should preferably be a non-signalling antibody, i.e. not induce activation of the signaling pathway downstream of the PDGFR.

To that end, they performed a screening and selection process of antibodies produced in a llama that was immunized with recombinant human PDGFR β ectodomain that had been preincubated with a sub-equimolar amount of its ligand PDGF-BB.

This approach resulted in the identification of a panel of antibodies capable of binding to immobilized PDGFR β with an unsurpassed apparent binding affinity in the low nanomolar range. Antibody-conjugation to a detectable label on the opposite site of the binding moiety did not affect the antigen binding properties. Moreover, the (conjugated) PDGFR β antibodies do not induce activation of AKT or ERK½ (at therapeutic or physiological) concentrations, indicating that high affinity binding was not accompanied by receptor activation. No binding to other receptors, e.g. (human) PDGFR α or EGFR was observed, at least at concentrations lower than 10⁻⁷ M. Fluorochrome-labeled antibody specifically accumulated in fibrotic tissue in mice as well as in fibrotic stroma of solid tumors in mice. Antibody-conjugation to liposomes allowed for targeting and uptake of the liposomes to PDGFR β-expressing cells.

Herewith, the invention provides PDGFR β-antibodies, in particular non-signaling PDGFR β targeting antibodies, that are very suitable for use in diagnostic and/or therapeutic targeting strategies, e.g. for targeting and inhibiting myofibroblasts.

In one embodiment, the invention provides an antibody that specifically binds to (PDGFR β) with a binding affinity of less than 10 nM, preferably less than 5 nM, more preferably less than 2 nM.

The term “binding affinity”, as used herein, includes the strength of a binding interaction and therefore includes both the actual binding affinity as well as the apparent binding affinity. The actual binding affinity is a ratio of the association rate over the disassociation rate. Apparent binding affinity is related to the association constant and dissociation constant for a pair of molecules, and relates to a non 1:1 or multivalent association between the pair of molecules. Apparent affinities as used herein to describe interactions between molecules of the described methods are observed in empirical studies, which can be used to compare the relative strength with which one molecule (e.g., an antibody or other specific binding partner) will bind two other molecules (e.g., two versions or variants of a peptide). The concept of binding affinity may be described as apparent Kd, apparent binding constant, EC50 or other measurements of binding. The apparent affinity can include, for example, the avidity of the interaction.

The term “PDGFR β”, as is used herein, refers to the platelet-derived growth factor receptor beta, a protein that in humans is encoded by the PDGFR β gene. The molecular mass of the mature, glycosylated PDGFR β protein is approximately 180 kDa. The gene is known as Ensembl: ENSG00000113721, Entrez Gene: 5159 and UniProtKB: P09619.

The antibody may specifically bind to monomeric PDGFR β and/or to dimeric forms of the PDGFR, of which at least one unit is PDGFR β. Preferably, the antibody binds to the dimeric form, since especially the dimeric PDGFR β are abundantly present in diseased fibrotic tissue or the stroma of malignant tumors. In one aspect, the antibody binds to the activated dimeric form of the PDGFR β with a binding affinity of less than 10 nM. In another aspect, it binds to the non-activated dimeric form of the PDGFR β with a binding affinity of less than 10 nM. This is particularly interesting when the antibody is used as an imaging diagnostic by targeting PDGFR β.

The PDGFR β protein is a typical receptor tyrosine kinase, which is a transmembrane protein consisting of an extracellular ligand binding domain, a transmembrane domain and an intracellular tyrosine kinase domain. It is found in the cell membrane of certain cell types, where it binds its ligand PDGF. This binding turns on (activates) the PDGFR β protein, which then activates other proteins inside the cell by adding a cluster of oxygen and phosphorus atoms (a phosphate group) at specific positions. This process, called phosphorylation, leads to the activation of a series of proteins in multiple signaling pathways.

The signaling pathways stimulated by the PDGFR β protein control many important processes in the cell such as growth and division (proliferation), movement, and survival. PDGFR β protein signaling is important for the development of many types of cells throughout the body.

As used herein, the term “non-signaling” means that the antibody has no detectable effect on downstream PDGFRβ signaling routes, be it agonistic or antagonistic signaling. In other words, the antibody does not interfere with receptor signaling. Hence, an antibody of the invention can be referred to as a “non-signaling-interfering PDGFR β antibody”.

In one embodiment, an antibody according to the invention is a non-agonistic PDGFR β antibody, meaning that its binding does not activate the (signaling downstream of) PDGFR β, i.e. it does not result in any detectable activation of the PDGFR β. The extent of PDGFR activation of is readily determined by methods known in the art. Suitable assays typically comprise analysis of (intracellular) protein kinase activity, for example ERK½ and AKT (Hua-Zhong Ying et al., 2017 (DOI: 10.3892/mmr.2017.7641).

The term “antibody” as used herein, refers to an antigen binding protein comprising at least a heavy chain variable region (VH) that binds to a target epitope. The term antibody includes monoclonal antibodies comprising immunoglobulin heavy and light chain molecules, single heavy chain variable domain antibodies, and variants and derivatives thereof, including scFv, tandem scFv, scFab, and improved scFab (Koerber et al., 2015. J Mol Biol 427: 576-86), chimeric variants of monoclonal and single heavy chain variable domain antibodies. The term also includes antibody mimetics such as a designed ankyrin repeat protein (i.e. DARPIN), a binding protein that is based on a Z domain of protein A, a binding protein that is based on a fibronectin type III domain (i.e. Centyrin), engineered lipocalin (i.e. anticalin), human IgG CH2 domain based binding proteins (i.e. Abdurin), human IgG CH3 domain based binding proteins (i.e. Fcab), and a binding protein that is based on a human Fyn SH3 domain (i.e. Fynomer) (Skerra, 2007. Current Opinion Biotechnol 18: 295-304; Skrlec et al., 2015. Trends Biotechnol 33: 408-418).

In a preferred embodiment, the invention provides a PDGFR β antibody comprising CDR1, CDR2 and CDR3 amino acid sequences as depicted in Table 1A.

A specific aspect of the invention relates to a PDGFR β antibody that comprises
a heavy chain CDR1, CDR2 and CDR3 sequence as defined by ID NO: 1, 5 and 9, respectively, or a variant sequence thereof showing at least 90%, preferably at least 95%, identity thereto;
a heavy chain CDR1, CDR2 and CDR3 sequence as defined by ID NO: 2, 6 and 10, respectively, or a variant sequence thereof showing at least 90%, preferably at least 95%, identity thereto;
a heavy chain CDR1, CDR2 and CDR3 sequence as defined by ID NO: 3, 7 and 11, respectively, or a variant sequence thereof showing at least 90%, preferably at least 95%, identity thereto; or
a heavy chain CDR1, CDR2 and CDR3 sequence as defined by ID NO: 4, 8 and 12, respectively, or a variant sequence thereof showing at least 90%, preferably at least 95%, identity thereto.

Variant sequences include conservatively substituted variants, which refer to an antibody comprising an amino acid residue sequence substantially identical to a sequence of a reference ligand of a target in which one or more residues have been conservatively substituted with a functionally similar residue and which displays the targeting activity as described herein. The phrase “conservatively substituted variant” also includes antibodies wherein a residue is replaced with a chemically derivatized residue, provided that the resulting peptide displays targeting activity as disclosed herein.

Examples of conservative substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; or the substitution of one acidic residue, such as aspartic acid or glutamic acid for another.

In one embodiment, a PDGFR β antibody of the invention comprises
a heavy chain CDR1, CDR2 and CDR3 sequence as defined by ID NO: 1, 5 and 9, respectively;
a heavy chain CDR1, CDR2 and CDR3 sequence as defined by ID NO: 2, 6 and 10, respectively;
a heavy chain CDR1, CDR2 and CDR3 sequence as defined by ID NO: 3, 7 and 11, respectively; or
a heavy chain CDR1, CDR2 and CDR3 sequence as defined by ID NO: 4, 8 and 12, respectively.

TABLE 1A
CDR sequences of anti-PDGRB antibodies
according to the invention.

| ID | Anti- | | |
|---|---|---|---|
| NO. | body | Identity | Sequence |
| 1 | 1B5 | Heavy | E/T S A M S |
| | | chain | |
| | | CDR1 | |
| 2 | 1H4 | Heavy | PFAMA |
| | | chain | |
| | | CDR1 | |
| 3 | 1D4 | Heavy | RDVVG |
| | | chain | |
| | | CDR1 | |
| 4 | 1E12 | Heavy | A/P Y L M/T G/S |
| | | chain | |
| | | CDR1 | |
| 5 | 1B5 | Heavy | G I S S G G G R S T T |
| | | chain | |
| | | CDR2 | |
| 6 | 1H4 | Heavy | RISWTAGRTE |
| | | chain | |
| | | CDR2 | |
| 7 | 1D4 | Heavy | LVTRSYSTY |
| | | chain | |
| | | CDR2 | |
| 8 | 1E12 | Heavy | A W R W P A T S T/S T Y |
| | | chain | |
| | | CDR2 | |
| 9 | 1B5 | Heavy | T G Y C S G Y N |
| | | chain | C N F A P |
| | | CDR3 | |
| 10 | 1H4 | Heavy | RFVPSSGPTVEDT |
| | | chain | |
| | | CDR3 | |
| 11 | 1D4 | Heavy | DQGFCSGSGCYDWRTFHV |
| | | chain | |
| | | CDR3 | |
| 12 | 1E12 | Heavy | R R G/E I L Y G P |
| | | chain | A P T S P/E T A |
| | | CDR3 | P Y D F |

In a preferred embodiment, the invention provides a PDGFR β antibody comprising the combination of CDR1, CDR2 and CDR3 amino acid sequences as depicted in Table 1B (see also FIG. 1). More specifically, the invention provides a non-signaling PDGFR β targeting antibody comprising:
a heavy chain CDR1, CDR2 and CDR3 sequence as defined by ID NO: 13, 17 and 21, respectively;
a heavy chain CDR1, CDR2 and CDR3 sequence as defined by ID NO: 14, 18 and 22, respectively;
a heavy chain CDR1, CDR2 and CDR3 sequence as defined by ID NO: 15, 19 and 23, respectively; or
a heavy chain CDR1, CDR2 and CDR3 sequence as defined by ID NO: 16, 20 and 24, respectively.

| ID | Anti- | | |
|---|---|---|---|
| NO. | body | Identity | Sequence |
| 13 | 1B5 | Heavy | E S A M S |
| | | chain | |
| | | CDR1 | |
| 14 | 1H4 | Heavy | PFAMA |
| | | chain | |
| | | CDR1 | |
| 15 | 1D4 | Heavy | RDVVG |
| | | chain | |
| | | CDR1 | |
| 16 | 1E12 | Heavy | A Y L M G |
| | | chain | |
| | | CDR1 | |
| 17 | 1B5 | Heavy | G I S S G G |
| | | chain | G R S T T |
| | | CDR2 | |
| 18 | 1H4 | Heavy | RISWTAGRTE |
| | | chain | |
| | | CDR2 | |
| 19 | 1D4 | Heavy | LVTRSYSTY |
| | | chain | |
| | | CDR2 | |
| 20 | 1E12 | Heavy | A W R W P T |
| | | chain | S T T Y |
| | | CDR2 | |
| 21 | 1B5 | Heavy | T G Y C S |
| | | chain | G Y N C N F A P |
| | | CDR3 | |
| 22 | 1H4 | Heavy | RFVPSSGPTVEDT |
| | | chain | |
| | | CDR3 | |
| 23 | 1D4 | Heavy | DQGFCSGSGCYD |
| | | chain | WRTFHV |
| | | CDR3 | |
| 24 | 1E12 | Heavy | R R G I L |
| | | chain | Y G P A T S P T |
| | | CDR3 | A Y D F |

Highly preferred is a PDGFR β antibody comprising the CDR's of antibody “1B5” comprising the heavy chain CDR1, CDR2 and CDR3 sequences as defined by ID NO: 13, 17 and 21, respectively.

In a preferred aspect, an antibody of the invention comprises a heavy chain variable region comprising a sequence as set forth in Table 1C ID NO: 25-32, or a variant sequence thereof. The variant sequence may show at least 90%, 91%, 92%, 93%, 94%, 95%, 96%, 97%, 98% or 99% sequence identity with a sequence of Table 1C. For example, the variant is a human heavy chain variable domain equivalent of an antibody herein disclosed. A humanized VHH antibody may comprise one or more amino acid substitutions. General strategies to humanize a camelid single-domain antibody are known in the art. See for example Vincke et al. (2009, J. Biol. Chem. 284, 3273-3284). Very good results can be obtained with an antibody comprising a heavy chain variable region comprising a sequence as defined in SEQ ID NO: 25 or 26.

The sequences with the pairs ID NO: 25/26; 27/28; 29/30 and 31/32 differ only with respect to the N-terminal amino acid residue. The sequence according to SEQ ID NO: 25, 27, 29 or 31 comprises an N-terminal Asp residue, which is particularly suitable for expression in a yeast host cell, e.g. S. cerevisiae. The sequence with ID NO: 26, 28, 30 or 32 (indicated as 1B5*, 1D4*, 1H4* and 1E12*, respectively) comprises an N-terminal Glu residue, which is particularly suitable for expression in a bacterial host cell, e.g. E. coli.

TABLE 1C
Heavy chain variable region
sequences of preferred antibodies
against PDGFR beta

| | | | |
|---|---|---|---|
| 25 | 1B5 | Heavy | EVQLVESGGGLVQPGGSLRLSCVASGF |
| | | chain | SFSESAMSWVRQAPGKGLEWLSGISSG |
| | | | GGRSTTYAESVEGRFTISRDDAKNTLM |
| | | | LQMNSLKSEDTAVYYCAKTGYCSGYN |
| | | | CNFAPRGQGTQVTVSS |
| 26 | 1B5* | Heavy | DVQLVESGGGLVQPGGSLRLSCVASGF |
| | | chain | SFSESAMSWVRQAPGKGLEWLSGISSG |
| | | | GGRSTTYAESVEGRFTISRDDAKNTLM |
| | | | LQMNSLKSEDTAVYYCAKTGYCSGYN |
| | | | CNFAPRGQGTQVTVSS |
| 27 | 1D4 | Heavy | EVQLVESGGGLVQAGDSLRLSCAVSGR |
| | | chain | AQSRDVVGWFRQAPGKEREFLALVTRS |
| | | | YSTYYGASVKGRFTISRDNAKNTVHLE |
| | | | MNSLKPEDTGVYYCAADQGFCSGSGC |
| | | | YDWRTFHVWGQGTQVTVSS |
| 28 | 1D4* | Heavy | DVQLVESGGGLVQAGDSLRLSCAVSGR |
| | | chain | AQSRDVVGWFRQAPGKEREFLALVTRS |
| | | | YSTYYGASVKGRFTISRDNAKNTVHLE |
| | | | MNSLKPEDTGVYYCAADQGFCSGSGC |
| | | | YDWRTFHVWGQGTQVTVSS |
| 29 | 1H4 | Heavy | EVQLVESGGGLVQPGGSLRVSCSASWR |
| | | chain | TGNPFAMAWFRQAPGKERELIARISWT |
| | | | AGRTEYAESAKGRFTISRDVAKKTMYL |
| | | | QMNSLTPEDTGVYFCAARFVPSSGPTV |
| | | | FDTWGQGTQVTVSS |
| 30 | 1H4* | Heavy | DVQLVESGGGLVQPGGSLRVSCSASWR |
| | | chain | TGNPFAMAWFRQAPGKERELIARISWT |
| | | | AGRTEYAESAKGRFTISRDVAKKTMYL |
| | | | QMNSLTPEDTGVYFCAARFVPSSGPTV |
| | | | FDTWGQGTQVTVSS |
| 31 | 1E12 | Heavy | EVQLVESGGGLVQAGGSLRLSCAPSER |
| | | chain | SFSAYLMGWFRQAQGKEREFVA |
| | | | AWRWPTSTTYYSDSGKGRFTITGDN |
| | | | AKNTVELEMNSLKPEDTAVYYCAA |
| | | | RRGILYGPATSPTAYDFWGQGTQ |
| | | | VTVSS |
| 32 | 1E12* | Heavy | DVQLVESGGGLVQAGGSLRLSCAPSER |
| | | chain | SFSAYLMGWFRQAQGKEREFVA |
| | | | AWRWPTSTTYYSDSGKGRFTITG |
| | | | DNAKNTVELEMNSLKPEDTAVYYCAA |
| | | | RRGILYGPATSPTAYDFWGQGTQ |
| | | | VTVSS |

The invention specifically relates to VHH antibodies against human PDGFR beta. The term “VHH antibody”, as is used herein, refers to an antibody that comprises at least one single heavy chain variable domain. In a specific aspect, the VHH antibody according to the invention is a single heavy chain variable domain antibody devoid of light chains, also referred to in the art as Nanobody™. Preferably, a VHH is an antibody of the type that can be found in Camelidae which are naturally devoid of light chains, or a synthetic VHH which can be constructed accordingly. For example, said VHH antibody may comprise camelid or humanized amino acid sequences and may be coupled, for example, to human or humanized Fc regions.

The term “Camelidae”, as is used herein, includes reference to Llamas such as, for example, Lama glama, Lama vicugna (Vicugna vicugna) and Lama pacos (Vicugna pacos), and to Camelus species including, for example, Camelus dromedarius and Camelus bactrianus).

As described herein, the amino acid sequence and structure of a heavy chain variable domain, including a VHH, can be considered to be comprised of four framework regions or ‘FR’, which are referred to in the art and herein as ‘Framework region 1’ or ‘FR1’; as ‘Framework region 2’ or ‘FR2’; as ‘Framework region 3’ or ‘FR3’; and as Framework region 4′ or ‘FR4’, respectively; which framework regions are interrupted by three complementary determining regions or CDRs, which are referred to in the art as ‘Complementarity Determining Region 1’ or ‘CDR1’; as ‘Complementarity Determining Region 2’ or ‘CDR2’; and as ‘Complementarity Determining Region 3’ or ‘CDR3’, respectively.

By way of example, VHH domains of the presently disclosed subject matter are included in Table 1C. Hence, also provided herein is a VHH antibody comprising a sequence as set forth in any one of ID NO: 25-32, or a variant sequence thereof.

In one aspect, the invention provides a VHH antibody herein referred to as “QPD-1B5” (abbreviated to “1B5”) according to ID NO: 25 or 26. This antibody was found to have an apparent binding affinity to PDGFR β of around 1 nM. Moreover, it was readily provided with a diverse set of useful moieties, including biotin, fluorophores, chelators and optical imaging dyes.

SPR analysis revealed a high affinity to the human PDGFR β ectodomain and high K-on, but a very low (not measurable) K-off. The calculated K_{D} is around 4-5 pM.

In another aspect, the invention provides a VHH antibody herein referred to as “QPD-1D4” (abbreviated to “1D4”) according to ID NO: 27 or 28. This antibody was found to have an apparent binding affinity to PDGFR β of less than 1 nM (around 0.5 nM). Moreover, it was readily provided with a diverse set of useful moieties, including biotin, fluorophores, chelators and optical imaging dyes. SPR analysis revealed a high affinity and high K-on, and a very low K-off. The calculated KD is around 20 pM.

In yet another aspect, the invention provides a VHH antibody herein referred to as “QPD-1H4” (abbreviated to “1H4”) according to ID NO: 29 or 30. This antibody was also found to have an apparent binding affinity to PDGFR β of less than 1 nM, and was readily provided with a diverse set of useful molecules. SPR analysis revealed a high affinity and high K-on, and a measurable K-off. The calculated KD is in the low nanomolar to high picomolar range, depending on the buffer composition.

Still further, the invention provides a VHH antibody herein referred to as “QPD-1E12” (abbreviated to “1E12”) according to ID NO: 31 or 32. This antibody was found to have an apparent binding affinity to PDGFR β of around 10 nM. It was readily provided with a diverse set of useful molecules, including biotin, fluorophores, chelators and optical imaging dyes. SPR analysis revealed a calculated KD of around 100 nM.

Antibodies of the presently disclosed subject matter also include amino acid sequences comprising one or more additions and/or deletions or residues relative to the sequence of a VHH domain, such as those whose sequence is disclosed herein, so long as the requisite targeting activity of the peptide is maintained. The term “fragment” refers to an amino acid residue sequence shorter than that of a sequence of the presently disclosed subject matter, e.g. VHH domains, or of a wild-type or full-length sequence.

Additional residues can also be added at either terminus for the purpose of providing a “linker” by which the VHH domains of the presently disclosed subject matter can be conveniently affixed or conjugated to a (detectable) label, solid matrix, or carrier. Amino acid residue linkers are usually at least one residue and can be 40 or more residues, more often 1 to 10 residues. Typical amino acid residues used for linking are tyrosine, cysteine, lysine, glutamic and aspartic acid, or the like. In addition, a peptide can be modified by terminal-NH₂ acylation (e.g., acetylation, or thioglycolic acid amidation) or by terminal-carboxylamidation (e.g., with ammonia, methylamine, and the like terminal modifications). Terminal modifications are useful, as is well known, to reduce susceptibility by proteinase digestion, and therefore serve to prolong the half life of the antibodies in solutions, particularly biological fluids where proteases can be present.

In one embodiment, the antibody of the invention comprises an N- and/or a C-terminal peptide-tag. For example, it comprises a tag that allows for site-specific antibody conjugation. Of particular interest is a peptide-tag comprising a Cys-residue. Other useful tags include those allowing for targeting and/or retention in an organ of interest. In a specific aspect, the antibody comprises a tag which enhances retention of the antibody in the kidney, see Huyvetter et al., Theranostic, 2014 Apr. 25;4(7):708-20.

In some embodiments, the VHH domains of the presently disclosed subject matter can be in the form of dimers and in some embodiments other multimeric formations. In some embodiments tumor accumulation of a small antibody is improved by increasing its molecular weight by dimerization. In addition to making homodimeric constructs, heterodimeric or multimeric constructs comprising two or more different VHH domains can be constructed in some embodiments. In some embodiments, in order to confer conformational flexibility on the molecule, two or more domains can be connected by a linker.

As will be appreciated by a person skilled in the art, an antibody according to the invention is suitably applied in in vivo imaging, diagnostics and/or therapy. To that end, the antibody preferably comprises one or more moieties that enable or facilitate such application(s). In one embodiment, the antibody comprises a detectable label, a therapeutic agent, a carrier, a moiety to modify the in vivo pharmacokinetic properties, or any combination thereof.

Exemplary detectable labels include in vivo detectable labels, preferably a detectable label which can be detected using nuclear magnetic resonance (NMR) imaging, near-infrared imaging, positron emission tomography (PET), scintigraphic imaging, ultrasound, or fluorescence analysis. The detection label can be coupled or bound to the antibodies according to the present inventions, directly (by covalent attachment/conjugation) or indirectly via a coupler, linker or chelator known in this field.

For example, the invention relates to a PDGFR β antibody comprising the NMR-nuclide 69-gallium or 71-gallium. Preferred nuclides are those available for antibody-based nuclear imaging, and include the PET imaging nuclides 18-F, 89-Zr, 68-Ga, 124-1, 64-Cu and 86-Y, and the SPECT imaging nuclides 111-In, 131-I, 123-I and 99m-Tc.

In a specific aspect, the invention provides a PDGFR β antibody, preferably a VHH antibody as herein disclosed, that is conjugated to an 18F-based radiotracer suitable for PET imaging. See for example Alauddin (Am J Nucl Med Mol Imaging. 2012; 2(1): 55-76).

18F-labeling of an antibody of the present invention can be achieved by methods known in the art, preferably using mild conditions. For example, Cu-catalyzed azide-alkyne cycloaddition (CuAAC) and several copper-free click reactions represent such methods for radiolabeling of sensitive molecules. Kettenbach et al. (BioMed Research International, vol. 2014, Article ID 361329) provide an overview about the development of novel 18F-labeled prosthetic groups for click cycloadditions and describe copper-catalyzed and copper-free click 18F-cycloadditions.

Fluorescently labeled antibodies are also emerging as a powerful tool for cancer localization in various clinical applications. Fluorescent probes are largely non-toxic and have been widely used in the clinical setting (indocyanine green, ICG) with very limited toxicity in humans. However, limitations in ICG use, such as low quantum yield and absence of a bioactive group for conjugation, have led to the exploration of alternate dyes to ensure consistent drug manufacturing and superior performance. These include Cy5/7 dyes, ICG, Fluorescein (FITC) and IRDye700/800. Antibody-conjugated fluorophores can be visualized either in the visible spectrum, such as fluoresceine isothiocyanate (FITC), or in the near-infrared (NIR) spectral range, including known NIR fluorescent dyes such as IRDye800CW.

Suitable radionuclides for antibody loading or conjugation are known in the art. In one embodiment, the radionuclide is selected from the group consisting of 111In, 111At, 177Lu, 211Bi, 212Bi, 213Bi, 211At, 62Cu, 67Cu, 90Y, 1251, 1311, 1331, 32P, 33P, 47Sc, 111Ag, 67Ga, 68Ga, 153Sm, 161Tb, 152Dy, 166Dy, 161Ho, 166Ho, 186Re, 188Re, 189Re, 211Pb, 212Pb, 223Ra, 225Ac, 77As, 89Sr, 99Mo, 105Rh, 149Pm, 169Er, 194Ir, 58Co, 80mBr, 99mTc, 103mRh, 109Pt, 119Sb, 189mOs, 192Ir, 219Rn, 215Po, 221Fr, 255Fm, 11C, 13N, 150, 75Br, 198Au, 199Au, 224Ac, 77Br, 113mIn, 95Ru, 97Ru, 103Ru, 105Ru, 107Hg, 203Hg, 121mTe, 122mTe, 125mTe, 227Th, 165Tm, 167Tm, 168Tm, 197Pt, 109Pd, 142Pr, 143Pr, 161Tb, 57Co, 58Co, 51Cr, 59Fe, 75Se, 201Tl, 76Br and 169Yb.

Methods for radionuclide labeling of an PDGFR β antibody so as to be used in accordance with the disclosed methods are known in the art. For example, a targeting molecule can be derivatized so that a radioisotope can be bound directly to it (Yoo et al. (1997) J Nucl Med 38:294-300). Alternatively, a linker can be added to enable conjugation. Representative linkers include diethylenetriamine pentaacetate (DTPA)-isothiocyanate, succinimidyl 6-hydrazinium nicotinate hydrochloride (SHNH), and hexamethylpropylene amine oxime (HMPAO) (Chattopadhyay et al. (2001) Nucl. Med. Biol. 28:741-744; Sagiuchi et al. (2001) Ann. Nucl. Med. 15:267-270; Dewanjee et al. (1994) J. Nucl. Med. 35:1054-1063; U.S. Pat. No. 6,024,938). Additional methods can be found in U.S. Pat. No. 6,080,384; Hnatowich et al. (1996) J. Pharmacol. Exp. Ther. 276:326-334; and Tavitian et al. (1998) Nat. Med. 4:467-471.

In one embodiment, the invention provides a compound of the general formula M-L-Q, wherein M is a diagnostic or therapeutic agent, L is a linker, and Q is a PDGFR β targeting (VHH) antibody as herein disclosed. In one embodiment, M may be a metal chelator in the form complexed with a metal radionuclide or not. Alternatively, M may be a radioactive halogen instead of a metal chelator. The metal chelator M may be any of the metal chelators known in the art for complexing with a medically useful metal ion or radionuclide. Preferred chelators include DTPA, DOTA, DO3A, HP-D03A, EDTA, TETA, EHPG, HBED, NOTA, DOTMA, TETMA, PDTA, TTHA, LICAM, MECAM, or peptide chelators. The metal chelator may or may not be complexed with a metal radionuclide, and may include an optional spacer such as a single amino acid. Preferred metal radionuclides for scintigraphy or radiotherapy include 99mTc, 51Cr, 67Ga, 68Ga, 47Sc, 51Cr, 167Tm, 141Ce, 111In, 168Yb, 175Yb, 140La, 90Y, 88Y, 153Sm, 166Ho, 165Dy, 166Dy, 62Cu, 64Cu, 67Cu, 97Ru, 103Ru, 186Re, 188Re, 203Pb, 211Bi, 212 Bi, 213Bi, 214Bi, 105Rh, 109Pd, 117mSn, 149Pm, 161Tb, 177Lu, 198Au, and 199Au. The choice of metal will be determined based on the desired therapeutic or diagnostic application. For example, for diagnostic purposes the preferred radionuclides include 64Cu, 67Ga, 68Ga, 99mTc, and 111In, with 99mTc and 111In being particularly preferred. For therapeutic purposes, the preferred radionuclides include 64Cu, 90Y, 105Rh and 90Y, 111In, 117mSn, 149Pm, 153Sm, 161Tb, 166Dy, 166Ho, 175Yb, 177Lu, 186/188Re, and 199Au, with 177Lu, 90Y, 186Re and 188Re being particularly preferred.

When the labeling moiety is a radionuclide, stabilizers to prevent or minimize radiolytic damage, such as ascorbic acid, gentisic acid, or other appropriate antioxidants, can be added to the composition comprising the labeled targeting molecule.

Where M is a diagnostic moiety, preferred diagnostic moieties include, for example, agents which enable detection of the compounds by such techniques as x-ray, magnetic resonance imaging, ultrasound, fluorescence and other optical imaging methodologies. A particularly preferred diagnostic moiety is a photolabel.

The antibody may comprise a therapeutic agent, preferably a therapeutic agent selected from the group consisting of a radionuclide, a cytotoxin, and a chemotherapeutic agent. In one embodiment, the antibody is conjugated to a drug, a prodrug, a toxin, an enzyme, a tyrosine kinase inhibitor, a sphingosine inhibitor, an immunomodulator, a cytokine, a hormone, a second antibody, a second antibody fragment, an immunoconjugate, a radionuclide, an antisense oligonucleotide, an RNAi, an anti-angiogenic agent, a pro-apoptosis agent, an anti-tumor agent, a cytotoxic agent, or any combination thereof.

Exemplary anti-tumor agents that can be conjugated to the PDGFR β antibody disclosed herein and used in accordance with the therapeutic methods of the presently disclosed subject matter include alkylating agents such as melphalan and chlorambucil, vincaalkaloids such as vindesine and vinblastine, antimetabolites such as 5-fluorouracil, 5-fluorouridine and derivatives thereof.

In a preferred aspect, an antibody of the invention is conjugated to a nuclide that allows for application of the antibody in targeted radionuclide therapy (TRNT). TRNT is a systemic treatment that aims to deliver cytotoxic radiation to cancer cells, with minimal exposure to healthy tissue. See Dhuyvetter et al. D'Expert Opin Drug Deliv. 2014 Dec 1; 11(12): 1939-1954. TRNT involves the use of a radio-labelled biologic or other vehicle to target and deliver a cytotoxic amount of radiation to inoperable or disseminated disease by emitting Auger, β- or α-particles. Exemplary nuclides for radiotherapy include 177-Lutetium, Radium-223 and Iodine-131.

The antibody may be attached to a carrier, preferably a drug carrier. For example, the carrier is selected from the group consisting of a liposome, a nanoparticle, a polymersome and a microcapsule. In one embodiment, the antibody is coated onto a nanoparticle comprising a substance of interest, preferably nanoparticles comprising a therapeutic agent.

In another embodiment, the PDGFR β-antibody is attached to a liposome to allow for liposomal targeting to PDGFR β-expressing tissue. Their biocompatibility, biodegradability, low toxicity, and capacity to encapsulate a vast variety of drugs make liposomes highly attractive as therapeutic drug carriers. Since phospholipid-based liposomes were first described, the targeting and delivery of therapeutic drugs and imaging agents using liposome nanocarriers have made significant advances. Progress in liposomal design is leading to improved systems for therapeutic as well as diagnostic applications. Liposomes are increasingly being developed towards contrast-enhanced, cellular, and molecular MRI diagnostic agents. More importantly, clinical studies have confirmed the therapeutic properties of liposomes with the introduction of liposomal drug formulations for the treatment of several diseases.

In another embodiment, the antibody is coupled to a polymersome. Polymersomes architecturally resemble liposomes but are highly stable and can encapsulate larger amounts of hydrophilic drugs compared with micelles. This makes them particularly interesting for the delivery of cargo intracellularly or for the controlled release of drugs.

Well-established chemical reactions have been applied to attach different moieties to the lipid or to the surface of preformed carriers such as liposomes, for example amine-carboxylic acid conjugation, disulfide bridge formation, hydrazone bond formation, and the thiol-maleimide addition reaction yielding a thioester bond. The invention therefore also provides liposomes or polymersomes that are decorated with PDGFR β-targeting antibodies, preferably PDGFR β-targeting VHH antibodies as herein disclosed.

The therapeutic efficacy of some antibodies, such as single chain diabodies (scDb), tandem scFv (taFv) molecules or VHH antibodies, is hampered by the short serum half-life due to their small size. Thus, a (VHH) antibody of the present invention is suitably modified, e.g. by recombinant technology, to achieve an increased serum half-life and improved pharmacokinetics without impairing their target binding capability and efficacy. To solve this problem, long-circulating serum proteins, such as human serum albumin (HSA), which has a high affinity and a strong stability, and therefore, it has been widely used in therapy and diagnostic researches.

Accordingly, also provided herein is an antibody wherein the moiety to modify in vivo pharmaco-kinetics properties comprise a serum protein binding structure, or a structure to influence the lipophilicity or clearance by the liver and/or kidney. Half-life extension can for example be achieved through fusion to an anti-HSA antibody, by PEGylation or by fusion to an Fc domain. In one aspect, the invention provides a half-life extended VHH antibody showing high (low nanomolar) binding affinity to PDGFR β, consisting of two sequence-optimized variable domains of llama-derived VHH antibodies, one directed against PDGFR β and one directed against HSA, which may be genetically fused via an amino acid linker such as GGGGSGGGS.

The invention also provides a bispecific or multispecific binding compound comprising a PDGFR β antibody according to the invention. Included are bivalent bispecific and bivalent biparatopic binding compounds comprising a PDGFR β VHH of the invention.

The binding compound is for example a polypeptide comprising at least one or two (or more) PDGFR β nanobody(ies) as herein disclosed. The polypeptide may contain at least a first nanobody that binds to one PDGFR beta epitope and a second nanobody that binds to a different blood or plasma protein, peptide, or any constituent that affects the pharmacokinetic behavior in blood. It may comprise two or more coupled identical nanobodies that bind PDGFR β epitopes on different monomers/dimers, or two or more coupled different nanobodies that bind different PDGFR β epitopes on the same or on different monomers/dimers. In one aspect, the bispecific or multispecific binding compound comprises two or more coupled different nanobodies from which one at least binds a PDGFR β epitope and at least one binds a different tumor, stroma or fibrosis associated antigen or epitope.

It is therefore also within the scope of the invention that, where applicable, an antibody of the invention can bind to two or more antigenic determinants, epitopes, parts, domains, subunits or conformations of the PDGFR β. Also, for example, when PDGFR β exists in an activated conformation and in an inactive conformation, the antibody of the invention may bind to either one of these confirmation, or may bind to both these confirmations (i.e. with an affinity and/or specificity which may be the same or different). Preferably, an antibody of the invention binds to a conformation of growth factor receptors in which it is bound to a pertinent ligand, may bind to a conformation of growth factor receptors in which it not bound to a pertinent ligand, or may bind to both such conformations (again with an affinity and/or specificity which may be the same or different).

It is also expected that the antibody of the invention will generally bind to all naturally occurring or synthetic analogs, variants, mutants, alleles, parts and fragments of the PDGFR β; or at least to those analogs, variants, mutants, alleles, parts and fragments of growth factor receptors that contain one or more antigenic determinants or epitopes that are essentially the same as the antigenic determinant(s) or epitope(s) to which the antibody of the invention binds e.g. in wild type PDGFR β. It is also included within the scope of the invention that the antibody of the invention binds to some analogs, variants, mutants, alleles, parts and fragments of PDGFR β, but not to others.

It is within the scope of the invention that the antibody of the invention only binds to PDGFR β in its multimeric form, or to both the monomeric and the multimeric form. In such a case, the antibody may bind to the monomeric form with an affinity and/or specificity that is the same as, or different from, preferably higher than, the affinity and specificity with which the antibody of the invention binds to the multimeric form of PDGFR β.

The invention furthermore provides a nucleic acid molecule encoding an antibody according to the invention, optionally fused to any of the peptides or proteins as described herein above. The terms “nucleic acid molecule” or “nucleic acid” each refer to deoxyribonucleotides or ribonucleotides and polymers thereof in single-stranded or double-stranded form. Unless specifically limited, the term encompasses nucleic acids containing known analogues of natural nucleotides that have similar properties as the reference natural nucleic acid. The terms “nucleic acid molecule” or “nucleic acid” can also be used in place of “gene”, “cDNA”, or “mRNA”. Nucleic acids can be synthesized, or can be derived from any biological source, including any organism. Nucleic acids of the presently disclosed subject matter can be cloned, synthesized, recombinantly altered, mutagenized, or combinations thereof. Standard recombinant DNA and molecular cloning techniques used to isolate nucleic acids are known in the art. Site-specific mutagenesis to create base pair changes, deletions, or small insertions are also known in the art. See e.g., Sambrook & Russell (2001) Molecular Cloning: a Laboratory Manual, 3rd ed. Cold Spring Harbor Laboratory Press, Cold Spring Harbor, N.Y.

The invention provides vectors comprising a nucleic acid molecule of the invention. In one embodiment, the vector contains a nucleic acid molecule encoding a heavy chain of an anti-PDGFR β immunoglobulin. The invention also provides vectors comprising polynucleotide molecules encoding fusion proteins, modified antibodies, antibody fragments, and probes thereof. In order to express the heavy and/or light chain of the anti-PDGFR β antibodies of the invention, the polynucleotides encoding said heavy and/or light chains are inserted into expression vectors such that the genes are operatively linked to transcriptional and translational sequences. Expression vectors include plasmids, YACs, cosmids, retrovirus, EBV-derived episomes, and all the other vectors that the skilled man will know to be convenient for ensuring the expression of said heavy and/or light chains. The skilled man will realize that the polynucleotides encoding the heavy and the light chains can be cloned into different vectors or in the same vector. In a preferred embodiment, said polynucleotides are cloned in the same vector.

Polynucleotides of the invention and vectors comprising these molecules can be used for the transformation of a suitable mammalian host cell, or any other type of host cell known to the skilled person. Transformation can be by any known method for introducing polynucleotides into a cell host. Such methods are well known of the man skilled in the art and include dextran-mediated transformation, calcium phosphate precipitation, polybrene-mediated transfection, protoplast fusion, electroporation, encapsulation of the polynucleotide into liposomes, biolistic injection and direct microinjection of DNA into nuclei.

Still further, it relates to a method for producing the PDGFR β antibody, the method comprising expressing the encoding nucleic acid molecule, typically comprised in a suitable expression vector, in a relevant host cell and recovering the thus produced antibody from the cell. Isolated polypeptides and recombinantly produced polypeptides can be purified and characterized using a variety of standard techniques that are known to the skilled artisan. See e.g., Principles of Peptide Synthesis, 2nd rev. ed. Springer-Verlag, Berlin/New York; Ausubel (1995) Short Protocols in Molecular Biology, 3rd ed. Wiley, New York.

An antibody herein disclosed, in particular when conjugated to a therapeutic and/or diagnostic moiety, is advantageously contained in a therapeutic composition, a diagnostic composition, or a combination thereof. In one embodiment, the invention provides a therapeutic composition, a diagnostic composition, or a combination thereof, comprising one or more PDGFR β-targeting ligands comprising an antibody according to the invention, preferably an PDGFR β-VHH antibody as herein disclosed. In one aspect, it is used in a method of diagnosing a PDGF-mediated disease or medical condition in a mammal.

As will be appreciated by a person skilled in the art, a PDGFR β-VHH antibody (PDGFR β nanobody) as herein disclosed finds its use in a large variety of known and yet to be discovered nanobody-based diagnostic and therapeutic applications. These include nanobody-based delivery systems for diagnosis and targeted tumor therapy. See for example Hu et al. (Front. Immun. 2017, Vol. 8. Article 1442).

A therapeutic composition, a diagnostic composition, or a combination thereof, of the presently disclosed subject matter comprises in some embodiments a pharmaceutical composition that includes a pharmaceutically acceptable carrier. Suitable formulations include aqueous and non-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are SDS in the range of in some embodiments 0.1 to 10 mg/ml, in some embodiments about 2.0 mg/ml; and/or mannitol or another sugar in the range of in some embodiments 10 to 100 mg/ml, in some embodiments about 30 mg/ml; and/or phosphate-buffered saline (PBS). Any other agents conventional in the art having regard to the type of formulation in question can be used. In some embodiments, the carrier is pharmaceutically acceptable. In some embodiments the carrier is pharmaceutically acceptable for use in humans.

An antibody of the invention (or a composition comprising the same) finds its use as targeting agent, diagnostic agent, therapeutic agent or any combination thereof. For example, the invention provides a PDGFR β antibody for use in immune-PET imaging. Immuno-PET is the in vivo imaging and quantification of antibodies radiolabeled with positron-emitting radionuclides. These application-matched radionuclides are conjugated to chimeric, humanized, or fully human antibodies to provide real-time, target-specific information with high sensitivity. The antibody for use in immune-PET is suitably conjugated to one or more of copper-64 (64 Cu, t½=12.7 hr), yttrium-86 (86 Y, t½=14.7 hr), iodine-124 (124 I, t½=100.3 hr), zirconium-89 (89 Zr, t½=78.4 hr), gallium-68 (68 Ga, t½=1.13 hr) and fluorine-18 (18 F, t½=1.83 hr). 89 Zr may be considered a preferred positron emitter due to its compatible half-life, ideal physicochemical characteristics for protein conjugation, and availability

In another embodiment, the antibody is used in a method of treatment of a PDGF-mediated disease or medical condition in a mammal. For example, the PDGF-mediated disease or medical condition is cancer, restenosis, fibrosis, angiogenesis, renal disease or cardiovascular disease. In one embodiment, the disease is a chronic inflammatory disease, early or late fibrosis, fibrotic tumour, NASH/liver fibrosis, kidney fibrosis, pancreatic cancer or colon cancer, cardiac fibrosis, systemic sclerosis, Crohn's disease, Dupuytren's disease or arthrosis.

An exemplary therapeutic use is photodynamic therapy (PDT), in particular antibody-based photodynamic therapy, which is called photoimmunotherapy (PIT). This is a highly innovative novel approach designed to improve tumor selectivity. Every tumor has a unique biological profile, which includes the expression of different cell surface antigens. Targeting PDGFR β antigens by using antibodies of the invention as “vectors” for selective delivery of photosensitizers would thereby overcome the severe off-target toxic side effects of conventional PDT, increase therapeutic efficacy, and decrease morbidity.

Further therapeutic uses include those relying on one or more of the therapeutic agents as described herein above. A method for treatment may further comprises the administration of at least one further (chemo)therapeutic agent.

Suitable methods for administration of a therapeutic composition, a diagnostic composition, or combinations thereof of the presently disclosed subject matter include but are not limited to intravascular, subcutaneous, or intratumoral administration. Further, upon a review of the instant disclosure, it is understood that any site and method for administration can be chosen, depending at least in part on the species of the subject to which the composition is to be administered. For delivery of compositions to pulmonary pathways, compositions can be administered as an aerosol or coarse spray.

For therapeutic applications, a therapeutically effective amount of a composition of the presently disclosed subject matter is administered to a subject. A “therapeutically effective amount” is an amount of the therapeutic composition sufficient to produce a measurable biological response (e.g., a cytotoxic response, or tumor regression). Actual dosage levels of active ingredients in a therapeutic composition of the presently disclosed subject matter can be varied so as to administer an amount of the active compound(s) that is effective to achieve the desired therapeutic response for a particular subject. The selected dosage level will depend upon a variety of factors including the activity of the therapeutic composition, formulation, the route of administration, combination with other drugs or treatments, tumor size and longevity, and the physical condition and prior medical history of the subject being treated. In some embodiments of the presently disclosed subject matter, a minimal dose is administered, and dose is escalated in the absence of dose-limiting toxicity. Determination and adjustment of a therapeutically effective dose, as well as evaluation of when and how to make such adjustments, are known to those of ordinary skill in the art.

For diagnostic applications, a detectable amount of a composition of the presently disclosed subject matter is administered to a subject. A “detectable amount”, as used herein to refer to a diagnostic composition, refers to a dose of such a composition that the presence of the composition can be determined in vivo or in vitro. A detectable amount will vary according to a variety of factors, including chemical features of the antibody being labeled, the detectable label, labeling methods, the method of imaging and parameters related thereto, metabolism of the labeled antibody in the subject, the stability of the label (e.g. the half-life of a radionuclide label), the time elapsed following administration of an active agent and/or labeled antibody prior to imaging, the route of drug administration, the physical condition and prior medical history of the subject, and the size and longevity of the tumor or suspected tumor. Thus, a detectable amount can vary and can be tailored to a particular application.

### LEGEND TO THE FIGURES

FIG. 1: Amino acid sequences of exemplary anti-PDGFR β VHHs (Kabat numbering as applied for VHHs, according to Riechmann and Muyldermans (1999, J Immunol Methods 23: 25-38).

FIG. 2. Dose response ELISA showing the apparent binding affinities of 4 representative VHHs (clones 1B5, 1D4, 1E12 and 1H4) to immobilized human PDGFR β. Bound VHHs were detected using rabbit-anti-VHH, DARPO and OPD.

FIG. 3: Detection of HL488-conjugated VHH in SDS-PAGE gel. A total of 0.1 μg of conjugated VHH and a prestained MW ladder were run on a 15% SDS-PAGE gel. VHH-bound HL488 (top bands) and free HL488 (lower bands) was detected using a D-Digit fluorescence scanner (LiCOR). Lanes 1-4: gel filtrated batches; lanes 5-8: dialyzed batches.

FIG. 4: Binding analysis of purified VHHs and two batches of conjugated VHHs to immobilized recombinant PDGFR β using ELISA. Bound VHHs were detected using rabbit-anti-VHH (Cat. #QE19), followed by donkey-anti-rabbit-HRP and OPD as substrate. All VHH bound to PDGFR β with apparent binding affinities in the nanomolar range and there was no drastic reduction in apparent binding affinity observed upon conjugation.

FIG. 5: Binding analysis of purified VHHs and IRDye800CW- and NOTA-conjugated VHHs to immobilized recombinant PDGFR β using ELISA. Bound VHH were detected using rabbit-anti-VHH (Cat. #QE19), followed by donkey-anti-rabbit- HRP and OPD as substrate. All VHH bound to PDGFR-β with apparent binding affinities in the nanomolar range and there was no drastic reduction in apparent binding affinity observed upon conjugation.

FIG. 6: VHH 1B5-biotin and 1E12-biotin binding to either human or mouse PDGFR β extra-cellular domain (ECD). VHH's were captured on ECD-coated on Maxisorp wells. Bound VHH-biotin was detected using streptavidin-HRP using OPD as a substrate. Mean absorbance with individual measurements are plotted. Top panels: binding to human PDGFR β-ECD (hECD). Bottom panels: binding to mouse PDGFR β-ECD (mECD).

FIG. 7: Binding of VHHs to PDGFR β-expressing HEK293 cells. Panel A: 1B5-HL488 binding to cells with or without PDGFR β. PDGFR β-negative HEK293 or PDGFR β-expressing HEK293-PDGFR β cells were incubated with 0.01-1000 nM VHH 1B5-HL488 for 1 h on ice. N=1, with duplicates. Panel B: VHH uptake in HEK293-PDGFR β and HEK293 cells. Cells were incubated with 1 or 10 nM VHH for 1 h at 37° C.

FIG. 8: Uptake and binding of representative conjugated antibodies VHH-HL488 (10 nM for 1 h at 37° C.) to HEK293 (top panels) or HEK293-PDGFR β cells (bottom panels) analysed by FACS. Mean fluorescence intensity (MFI) of n=1 is also indicated.

FIG. 9: Uptake and binding of VHH-HL488 to HEK293-PDGFR β cells. Cells were incubated at 37° C. for 1 h and analysed for HL488. Panel A: % of HL488-positive cells with SEM. Panel B: Mean fluorescence intensity (MFI) of three independent experiments with SEM is shown.

FIG. 10. Binding vs. uptake of VHH-HL488 to PDGFR β expressing cells. Cells were incubated with 1 or 10 nM VHH-HL488 for 1 h at 37° C. or 4° C. Panel A: % of positive cells. Panel B: Mean fluorescence intensity (MFI) of three independent experiments with SEM is shown.

FIG. 11: VHH (flag-his-tagged, or conjugated with HL488, NOTA or 800 CW) binding to human extracellular domain (ECD) of PDGFR β assessed with surface plasmon resonance (SPR). Fc/His-tagged PDGFR β ECD was bound to protein A chemically conjugated to CM4 sensor chip. Binding of VHHs was tested at 0.39-50 nM. Shown are representative SPR Biacore traces of tagged or conjugated 1B5 VHH (panels A); tagged or conjugated 1D4 VHH (panels B); tagged or conjugated 1H4 VHH (panels C) or tagged VHH 1E12 (panel D).

FIG. 12: Assessment of pAKT and pERK½ signaling in response to VHH. HHsteC cells were serum-starved for 24h and then stimulated with 5 ng/ml of TGF-β for 24 h. pAKT or pERK½ was induced by 50 ng/ml PDGF-BB for 30 min as a positive control. Cells were incubated with VHHs 1B5, 1D4, 1H4 or 1E12 (flag-his-tagged) at 1 μM, 100 nM or 10 nM for 30 min at 37° C. prior to cell lysis.

FIG. 13: Quantification of pAKT and pERK½ band intensities, normalized to beta-actin. Actin primary antibodies (Sigma-Aldrich) were diluted in Odyssey Blocking Buffer (LI-COR) and incubated at 4° C. o/n, followed by secondary anti-mouse and anti-rabbit IRDye 680 RD and 800 CW antibodies (LI-COR). Signals were scanned on a Li-Cor Odyssey image scanner. Mean of 1-4 experiments with SEM is shown.

FIG. 14: Biotin-conjugated VHHs 1D4, 1H4, 1E12 or 1B5 do not bind human PDGFRa (panel A and B) or human EGFR (panels C and D). VHH binding to human PDGFRa/EGFR was assessed in a direct ELISA using PDGFRa/EGFR coating and biotin-streptavidin-HRP detection. ELISA method and the presence of PDGFRa/EGFR was confirmed with a positive control anti-PDGFRa/anti-EGFR antibody and an HRP-conjugated anti-rabbit antibody. Wells containing no primary antibody was used as a negative control. Mean of 3-6 experiments with duplicate wells are shown with SEM.

FIG. 15: VHH uptake in renal fibroblasts and myofibroblasts. Renal fibroblasts were stimulated with serum starvation and 5 ng/ml TGFbeta for 7 days to transform them into myofibroblasts. Cells were incubated with 10 nM VHH-HL488 for 1 h (panel A) or 24 h (panel B), and were analysed for fluorescence content using a plate reader. Mean of 3 independent measurements with SEM is shown. HEK293-PDGFRB was used as a positive control (n=1).

FIG. 16: Uptake vs binding of VHH-HL488 in serum-starved TGFbeta-stimulated HHSteC. Cells were serum-starved and TGFbeta-stimulated prior to VHH-HL488 treatment at 0.1-10 nM for 1 h at either 37° C. or 4° C. Cells were analysed on a FACS. Mean uptake of two independent experiments with SEM in TGFB-treated cells is shown (0. 1 nM VHH treatment n=1). Panel A: % of HL488-positive cells of living cells. Panel B: mean fluorescent intensity in living cells.

FIG. 17: VHH-mediated targeting and uptake of liposomes. Calcein-loaded liposomes conjugated to antibody 1B5 (VHH to liposome at ratio 3, 10, 30 and 100) are taken up and bound by cells expressing PDGFRβ. HEK293-PDGFRβ and HEK293 were incubated with liposome 1B5 or J3RSc constructs for 6 h at 37° C. or on ice for 6 h. Fluorescent signal of calcein in cells was measured using a plate reader. Liposome-0 incubated at 37° C. or on ice were used to normalize fluorescent signal. Mean of 2-5 independent experiments is shown with SEM. ***; P 0.0001-0.001, ****; P<0.0001 with 2-way ANOVA with Dunnett's post test, compared to liposome-0 control. All other differences are statistically not significant.

FIG. 18: Ex vivo Near Infrared imaging of conjugated PDGFRβ-VHHs in fibrotic tissue. Male C57BL/J6 mice received a single dose of Bleomycin intratracheally (0.08 mg/kg in 50 uL PBS). 3 weeks after the start of the bleomycin application, mice (right panel) were injected with 40 μg VHH-conjugate and whole animals were scanned 2-6 hours after probe injection using fluorescence mediated tomography. Control mice (left panel) received VHH J3RSc, that only binds to HIV. Immediately after the last in vivo scan, animals were euthanized and the lungs were excised and scanned ex vivo.

### EXPERIMENTAL SECTION

### Example 1: Production and Sequencing of PDGFR β-Antibodies.

Llama were immunized according to standard procedures with the extracellular domain (ECD) of recombinant PDGFR β protein. Prior to immunization, PDGFR β ECD was preincubated with PDGF-BB, wherein PDGFR β ECD was present in 5 times molar excess. It was estimated that PDGFR binders and PDGF-competing VHH should be able to be isolated from such a library. RNA from this animal was isolated from the PBMCs after the immunization protocol.

### Library Construction in Llama

CDNA synthesis

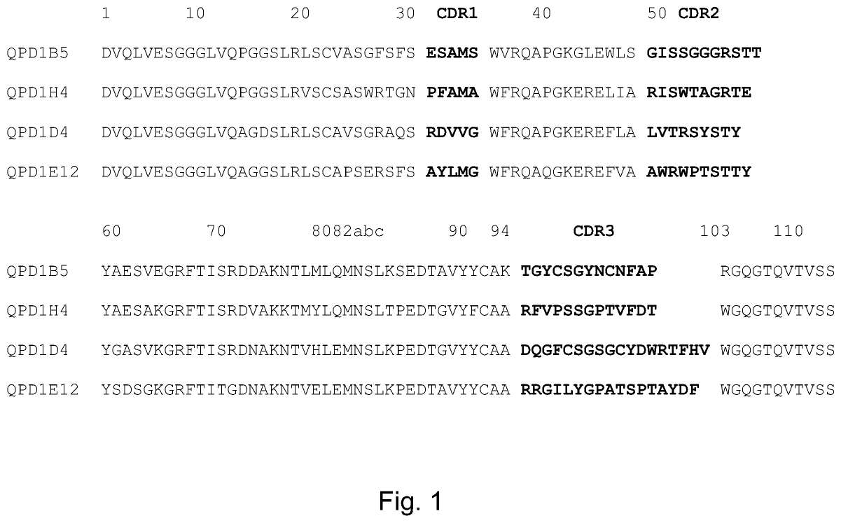

Intact 28S and 18S rRNA were clearly visible indicating proper integrity of the RNA. Precipitated RNA was dissolved in RNase-free MQ and the RNA concentrations were measured. About 40 μg RNA (4 reactions of 10 μg each) was transcribed into cDNA using a reverse transcriptase Kit (Invitrogen). The cDNA was purified on Macherey Nagel PCR clean-up columns. IG H (both conventional and heavy chain) fragments were amplified using primers annealing at the leader sequence region and at the CH2 region. 5 μl was loaded onto a 1% TBE agarose gel for a control of the amplification. FIG. 1 shows that the two DNA fragments (˜700 bp and ˜900 bp) were amplified representing the VHH and VH, respectively. After this control, the remaining of the samples were loaded on a 1% TAE agarose gel and the 700 bp fragment was excised and purified from the gel. About 80 ng was used as a template for the nested PCR (end volume 800 μl) to introduce SfiI and BstEII restriction sites. The amplified fragment was cleaned on Macherey Nagel PCR cleaning columns and eluted in 60 μl. The eluted DNA was digested with SfiI and BstEII. As a control of the restriction digestion, 4 μl of this mixture was loaded onto a 1.5% TBE agarose gel. After the restriction digestion, the samples were loaded on a 1.5% TAE agarose gel. The 400 bp fragment was excised from the gel and purified on Machery Nagel gel extraction columns. The purified 400 bp fragments (˜330 ng) were ligated into the phagemid pUR8100 vector (˜1 μg) and transformed into TG1. The transformed TG1 were titrated using 10-fold dilutions. 5 μl of the dilutions were spotted on LB-agar plates supplemented with 100 μg/ml ampicillin and 2% glucose to determine the library size. The number of transformants was calculated from the spotted dilutions of the rescued TGI culture (total end volume is 8 ml). The total number of transformants and thereby the size of the library was calculated by counting colonies in the highest dilution and using the formula below:

Library size=(amount of colonies)*(dilution)*8(ml)/0.005(ml; spotted volume).

All libraries were of good size with more than 10⁷ clones per library. The bacteria were stored in 2×YT medium supplemented with 20% glycerol, 2% glucose and 100 μg/ml ampicillin at −80° C.

Phage production and Selections

For the selections phages were produced according to SOP33. Titers of the libraries were all >10¹¹ per ml.

For the 1^{st} round of selections, 20 μl of the precipitated phages (corresponding to >1000-fold the diversity of the libraries) of each library were pre-blocked and applied to wells coated with PDGFR. For both libraries non-specific binding phages were eluted from the non-coated wells. Outputs of binding phages were eluted from the coated wells, there was a concentration dependent enrichment between the different concentrations visible (data not shown).

TG 1 cultures infected with the output of the selection on 5 μg/ml PDGFR β (highest coating) were used for phage production in order to perform the 2^{nd} round of selection. Input phages were as expected and controls were empty. For the 2^{nd} round of selection, 1 μl of the precipitated phages was applied to wells coated with PDGFR β, in which we used three concentrations of PDGFR β, for which the lowest concentration should result in the highest affinity binders.

Very high outputs were eluted from the coated wells, showing a concentration dependent enrichment between the different concentrations used, indicating that VHH were selected that bind specifically to PDGFR β.

After the 2^{nd} round of phage display selection, phages were rescued by infection of E. coli TG1 and glycerol stocks were prepared from all outputs. These were stored at −80° C. in the same way as for the outputs obtained after the 1^{st} round of phage display selection. Subsequently, rescued outputs of the 1st and of the 2^{nd} round of selection on PDGFR β were plated out in order pick single clones. For master plate QPD-1, a total of 92 single clones were picked in a 96-wells plate. In order to screen master plate QPD-1 for PDGFR βbinders, periplasmic extracts containing monoclonal VHH were produced. To test the binding specificity of the monoclonal VHH by ELISA, R β (2μg/ml PBS) was coated overnight onto Maxisorp plates at 4° C. Most of the clones from the Tilly library were able to bind specifically to PDGFR β. Some good binding VHH's were selected from the non immune library as well (data not shown).

### Sequence Analysis of VHHs

In order to determine the diversity of the selected VHH clones, a subset of binders of the Peri ELISA were picked and subjected to sequencing. FIG. 1 shows a sequence alignment of 4 different VHH sequences QPD-1B5, QPD-1D4, QPD-1E12, and QPD-1H4, which originated from three different germline families (KGLEW, KEREL and KEREF).

### Example 2: Dose Response ELISA

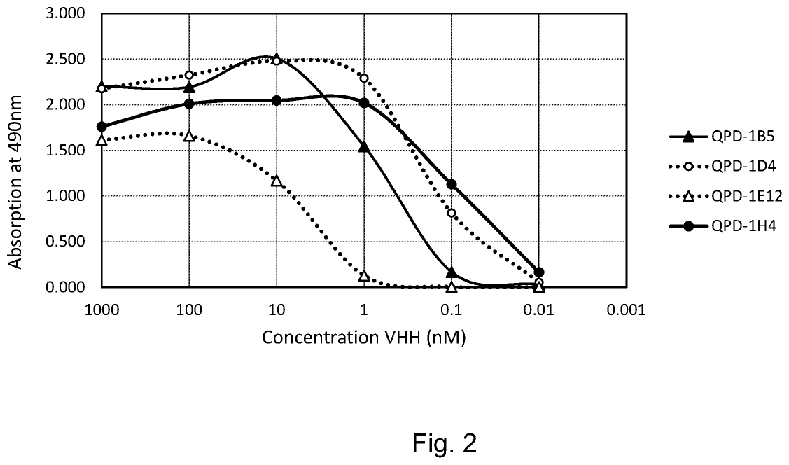

The apparent binding affinity of exemplary PDGFR β VHH's was tested in an ELISA using 96-well Maxisorp plates that were coated with 50 μl of 2 μg/ml PDGFR β ECD (U-protein Express, Utrecht) antigen in sterile PBS. A serial dilution of the VHHs was added to the coated wells and incubated for 1 hour at room temperature starting at 1000 nM. Bound VHH's were detected with rabbit anti-VHH, DARPO and made visible with OPD80. All VHH showed binding to the immobilized PDGFR antigen (see FIG. 2). Interestingly, QPD-1D4 and QPD-1H4 have an apparent affinity lower than 1 nM. The apparent affinity of QPD-1B5 is ˜1 nM and for QPD-1E12 the apparent affinity is around 10 nM.

The 4 clones that were tested were recloned in a suitable expression vectors for production in bacterial or yeast host cells according to published methods (Heukers et al. Antibodies 2019, 8(2), 26). For this, the VHH genes were cloned into the pMEK222 vector for production in E. coli, which provides the VHH with a C-terminal FLAG-His tag. VHHs were produced and purified from E. coli TG1 using immobilized metal-affinity chromatography (IMAC, Thermo Fisher Scientific Waltham, MA, USA).

For production in yeast, VHH genes were recloned in the pYQVQ11 vector for VHH production in yeast, which provides the VHH with a C-terminal C-Direct tag containing a free thiol (cysteine) and an EPEA (Glu, Pro, Glu, Ala) purification tag (C-tag, Thermo Fisher Scientific). To improve production yields and facilitate purification from supernatant, C-Direct-tagged VHH were produced in several 1 L S. cerevisiae cultures, and purified via affinity chromatography anti-EPEA (C-tag) columns of Thermofisher according to the manufacturer's protocols. Purified VHH was filter sterilized and stored in PBS.

### Example 3: Manufacture and Characterization of Conjugated Antibodies

This example describes the preparation and characterization of various VHH conjugates. VHHs were site-directionally conjugated to Biotin-maleimide (Pierce, ThermoFisherScientific), HiLyte Fluor 488-maleimide (Anaspec), IRDye800CW-maleimide (LI-COR Biosciences) or NOTA-maleimide chelator (Chematech) using methods known in the art (Heukers et al. Antibodies 2019, 8(2), 26).

First, the VHHs were incubated with an 2.75-fold molar excess of TCEP (tris(2-carboxyethyl) phosphine hydrochloride) (VWR International, Radnor, PA, USA) to reduce the C-terminal cysteine upon which the VHH were incubated with an excess of maleimide-conjugated labels for 2 h at 37° C.

Free label was removed by size-exclusion chromatography using two consequent Zeba Desalting Columns (ThermoFisherScientific) according to the manufacturer's protocols. For the fluorophorese, the degree of conjugation was determined using the Multiskan Go spectrophotometer (Thermo Fisher Scientific), and the amount of free dye was determined after size separation by SDS-PAGE (Bio-Rad) on a D-Digit or Odyssey scanner (Li-COR Biosciences). Afterwards, the SDS-PAGE gel was stained with Page Blue (Thermo Fisher Scientific) to show the integrity of the conjugated protein.

### Example 3A

This example describes the characterization of VHH conjugates to HiLyte-488 (HL488), which is a widely used fluorophore comparable to FITC and Alexa 488.

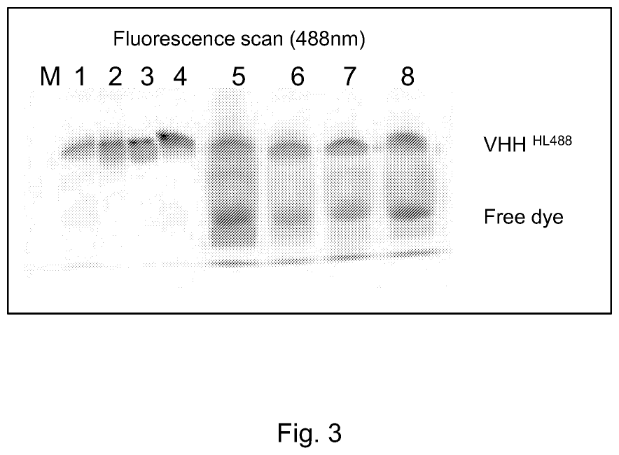

FIG. 3 shows SDS-PAGE analysis of HL488-conjugated VHH to determine the conjugation efficiency. A total of 0.1 μg of conjugated VHH (clones 1E12, 1B5, 1H4 and 1D4) and a prestained MW ladder were run on a 15% SDS-PAGE gel. VHH-bound HL488 (top bands) and free HL488 (lower bands) were detected using a D-Digit fluorescence scanner (LiCOR). The gel filtrated batches (lanes 1-4) only contained VHH-bound HL488, while there was still some free dye in the dialyzed batches (lanes 5-8). These data shown that all representative VHH were successfully conjugated to HL488.

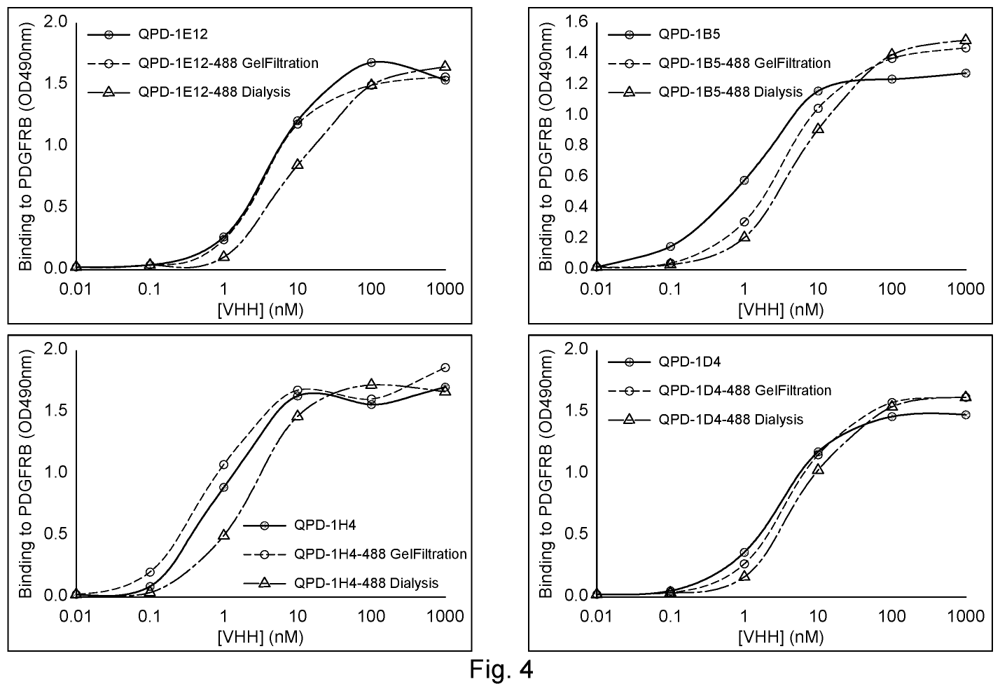

Next, the binding of the 4 purified QPD clones and the two batches of HL488-conjugated QPD clones to immobilized recombinant PDGFR-β was determined using an ELISA assay as described herein above. Bound VHH were detected using rabbit-anti-VHH (Cat. #QE19), followed by donkey-anti-rabbit-HRP and OPD as substrate. All (conjugated) VHH were found to bind to PDGFR-B with apparent binding affinities in the nanomolar range and no drastic reduction in apparent binding affinity upon conjugation was observed. See FIG. 4.

### Example 3B

This example describes the characterization of representative VHHs to the NOTA-maleimide chelator or to the near-infrared dye and IRDye-800CW, which is widely used in near-IR optical imaging.

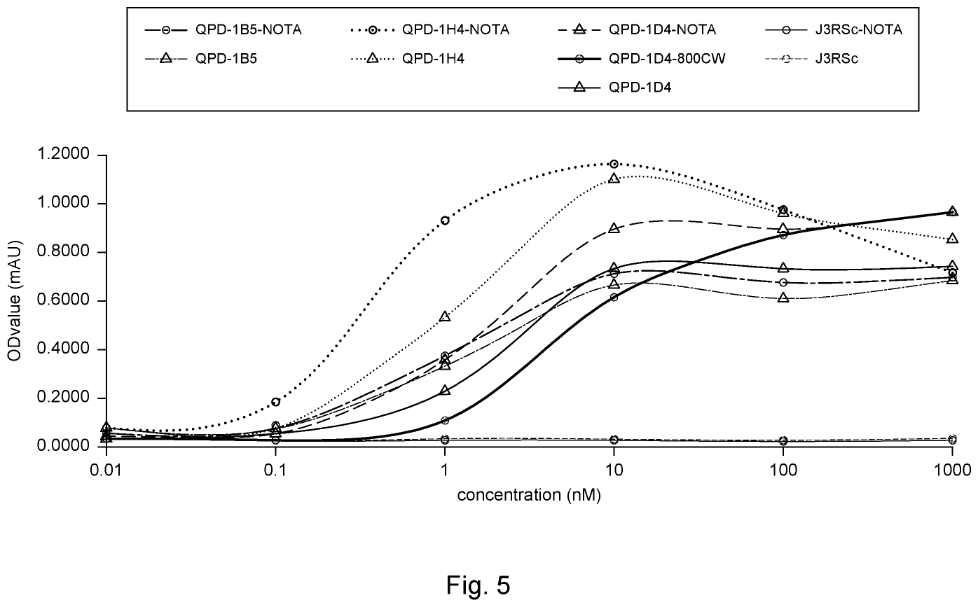

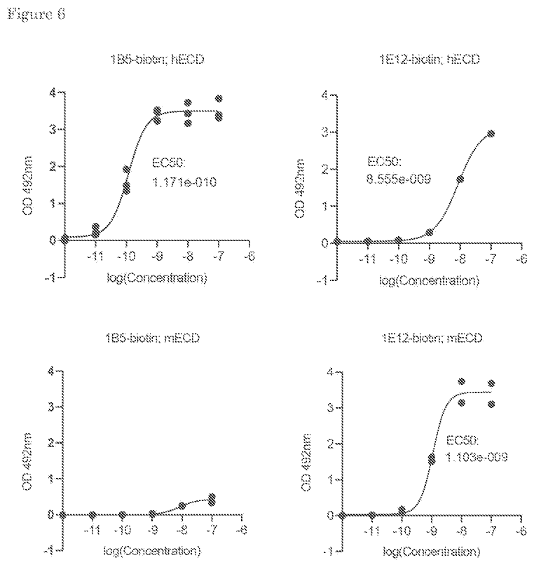

FIG. 5 shows the binding of either purified VHH clones, IRDye800CW- or NOTA-conjugated VHH clones to immobilized recombinant PDGFR-B using ELISA. Bound VHH were detected using rabbit-anti-VHH (Cat. #QE19), followed by donkey-anti-rabbitHRP and OPD as substrate.

All VHHs were found to bind to PDGFR-B with apparent binding affinities in the nanomolar range, and there was no drastic reduction in apparent binding affinity observed upon conjugation.

### Example 5: Species Cross-Reactivity

In this example, the binding affinity of biotin-conjugated VHHs 1B5 and 1E12 to either human or mouse PDGFR β extra-cellular domain (ECD) was assessed using biotin-streptavidin ELISA. Human or mouse PDGFR β ECD was coated on immunoassay wells, where 1B5-biotin or 1E12-biotin were captured. Binding was detected using streptavidin-HRP and ODP as a substrate.

ELISA Method 96-well maxisorp (Nunc) immunosorp plates were coated with 1 ug/ml or 2 ug/ml of PDGFR β extracellular domain (ECD; Fc- and His-tagged, Sino Biological) or mouse PDGFR β ECD (R&D Systems) 4 ug/ml in PBS, 100 ul per well, at 4C overnight. Wells were washed thrice in PBS. Unspecific binding sites were blocked in 4% BSA/PBS for 1h at RT, followed by washing thrice in PBS. VHHs 1B5-biotin or 1E12-biotin, diluted at 0.01-100 nM in 1% BSA/PBS, was allowed to bind on ECD for 1 h at RT, followed by washing thrice in PBS. Streptavidin-horseradish peroxidase (HRP; GeneTex) was diluted 1:10,000 in 1% BSA/PBS and was incubated for 1 h at RT. Wells were washed 6 times in PBS. o-Phenylene diamine dihydrochloride (OPD; Sigma-Aldrich) was used as HRP substrate at 0.4 mg/ml in 0.05 M phosphate-citrate buffer pH 5.0 with 0.03% sodium perborate (Sigma-Aldrich), prepared according to the manufacturer's recommendations, and was used in volume of 100 ul per well. Reaction was allowed to proceed for 30 min at RT in dark, and was stopped by adding 50 ul of 1.5 M HCl. Optical density was measured at 492 nm on a Synergy H1 microplate reader (BioTek).

### Data Analysis, Statistical Analysis

Absorbance from non-specific binding in the absence of PDGFR β ECD was subtracted from specific signal with PDGFR β ECD present. Mean absorbance from independent experiments (1B5-biotin on hECD n=3, 1E12-biotin on hECD n=1, on mECD n=2) with standard error of the mean (S.E.M.) was graphed on an XY plot using Prism 8 (GraphPad) software. Data were analyzed using nonlinear regression model and equation of log(inhibitor) vs. response, variable slope four parameters. Blanc absorbance value with 0 nM VHH-biotin was included as 10-12. Results are shown in FIG. 6.

VHH 1B5-biotin was captured on human PDGFR β ECD-coated (1 ug/ml) Maxisorp plates at 0.01-100 nM. Binding reached plateau at 1 nM of 1B5-biotin (log-9 molar). EC50 was determined as 1,171×10⁻¹⁰ M. VHH 1E12-biotin was captured on immobilized human PDGFR β ECD (2 ug/ml) at concentrations of 0.01-100 nM.

These data show that 1B5-biotin efficiently binds to human PDGFR β, while 1E12-biotin does so only moderately. 1B5-biotin affinity to mouse ECD was found weak. In contrast, 1E12-biotin cross-reacted with mouse ECD. 1E12-biotin at 10 nM reached the plateau of binding, and EC50 was determined as 1,1×10⁻⁹ M.

In conclusion, 1B5-biotin was found to strongly associate with human PDGFR β, with an EC50 of 1,171×10⁻¹⁰ M, while its affinity to mouse PDGFR β was poor. Interestingly, 1E12-biotin, in contrast, has a strong affinity to mouse PDGFR β, with an EC50 of 1,1×10⁻⁹ M, and a moderate affinity to human PDGFR β.

### Example 6: Antibody Binding to Mammalian Cells Expressing Human PDGFR β.

In order to demonstrate antibody binding activity to PDGFR β expressed on intact cells, the binding of 4 exemplary conjugated antibodies of the invention to human embryonic kidney (HEK293) cells that were stably transfected with PDGFR β was determined.

### Cells

HEK293 cells stably expressing human PDGFR β (HEK293-PDGFR β) were generated using an iDimerize inducible dimers system (Clontech Laboratories, Inc, a Takara Bio Company, JP) according to manufacturer's instructions. Cells were cultured in Dubecco's Modified Eagle Medium high glucose, supplemented with 10% FBS, 1% sodium pyruvate, 1% non-essential amino acids, 1% L-glutamine, 1% penicillin/streptomycin and 300 ug/ml hygromycin. HEK293 cells (control cells) were purchased from ECACC/Sigma Aldrich and were cultured in Dulbecco's Modified Eagle Medium high glucose (+10% FCS+1% penicillin/streptomycin+1% L-glutamin).

### Antibodies

Conjugated VHH-HL488 antibodies were produced as described above and purified using either gel-filtration or dialysis. Gel-filtrated batches are referred to as “VHH-HL488”, and the dialyzed batch is referred to as “VHH-HL488 dialyzed”.

| | Molarity | MW | Estimated free dye |
|---|---|---|---|
| VHH | (μM) | (Da) | (%) |
| 1B5-HL488 | 27.1 | 14769.3 | 0 |
| 1D4-HL488 | 9.1 | 15571.1 | 0 |
| 1H4-HL488 | 32 | 15073.8 | 0 |
| 1E12-HL488 | 11.9 | 15596.2 | 2 |
| 1B5-HL488 dialyzed | 116.2 | 14769.3 | 34 |
| 1D4-HL488 dialyzed | 14.5 | 15571.1 | 46 |
| 1H4-HL488 dialyzed | 67.5 | 15073.8 | 40 |
| 1E12-HL488 dialyzed | 26.5 | 15596.2 | 53 |

### Plate Reader Assay

Cells grown in flasks were detached by trypsin, counted and resuspended in 10% FBS/PBS and aliquoted in Eppendorf tubes containing 200,000 cells per treatment. Cells were incubated either on ice, at 4° C. or at 37° C. for at least 20 min prior to starting VHH treatment to obtain target temperature. VHH-HL488 was added in the cell suspension at 0.01-1000 nM and treatments were incubated for 1 h either on ice, at 4° C. or at 37° C. Cells were washed three times in cold PBS. Pelleted cells were resuspended in total of 200 ul of PBS and were loaded on black 96-well plates for fluorescent measurement at 488/530nm on a Synergy H1 microplate reader (BioTek), using a top measurement at gain of 100.

### FACS Assay

Cells were treated as per above at 0.01-1000 nM of VHH-HL488, either at 4° C. or 37° C. for 1 h in 10%FBS/PBS. Cells were washed twice in 2% FBS, 5 mM EDTA in PBS prior to adding PI at a final concentration of 0.1 ug/ml prior to analysis, also in 2% FBS, 5 mM EDTA in PBS. FACS was performed using the MacsQuant instrument. Data is presented either as % of HL488-positive cells in a live cell population or mean fluorescent intensity (MFI).

### Data Analysis, Statistical Analysis

Data from 3-× independent experiments are shown as mean, unless otherwise stated. Dialyzed batch: n=1−2.

### Results

### VHH-HL488 Uptake and Binding in HEK293-PDGFR β Analysed by Plate-Reader

VHH binding and uptake to PDGFR β was assessed using HEK293 cells that stably express PDGFR β (HEK293-PDGFR β). HEK293 cells lacking PDGFR β served as a negative control. First, cellular binding of VHH was assessed. To prevent VHH uptake and to allow binding to occur, cells were incubated with 1B5-HL488 on ice for 1 h. Unbound compound was removed by washing, and remaining fluorescence bound by cells was measured using a plate reader.

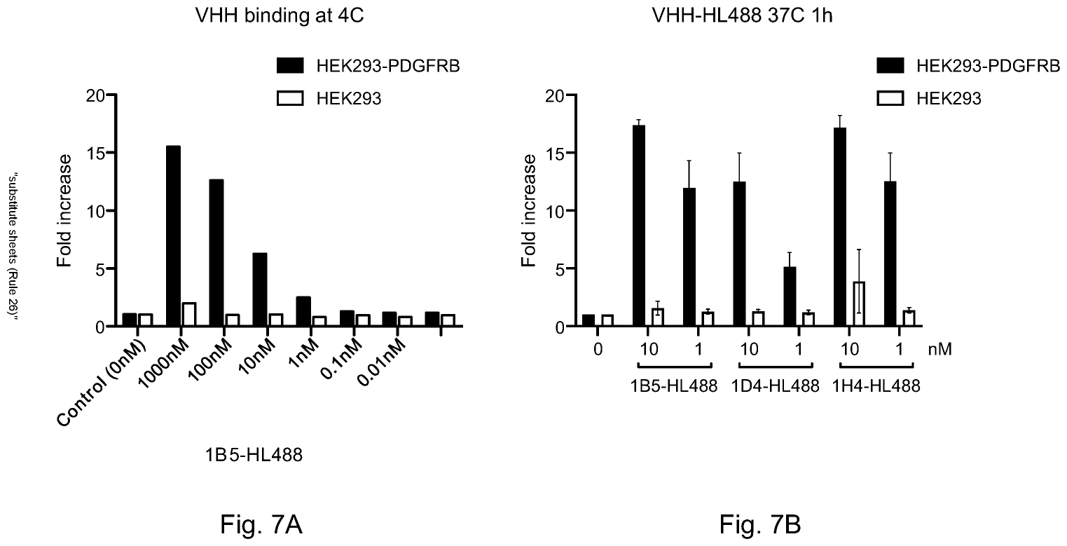

HEK293-PDGFR β cells exhibited a dose-dependent binding of 1B5-HL488, where 1 nM seemed a detection limit. At 1000 nM, the signal was not yet saturated. HEK293 control cells did not bind 1B5-HL488 (see FIG. 7A). These data indicate that 1B5-HL488 binds cells, and that cellular binding is dependent on PDGFR β.

Next, the uptake of a wider selection of VHHs (1B5-HL488, 1D4-HL488 and 1H4-HL488) was assessed in HEK293-PDGFR β and HEK293 cells. Incubation at 37° C. allows cells to bind VHHs as well as to possibly internalize VHHs. Cells were incubated at 10 nM or 1 nM of VHHs for 1 h. After washing, remaining fluorescence in cells was measure using the plate reader. HEK293-PDGFR β effectively took up and bound all tested VHHs. In contrast, HEK293 did not take up any of the tested VHHs (see FIG. 7B). This demonstrates that HL488-tagged 1B5, 1D4 and 1H4 are bound and/or taken up by cells, and that uptake is dependent on PDGFR β.

### VHH-HL488 Uptake and Binding to HEK293-PDGFR β Analysed by FACS

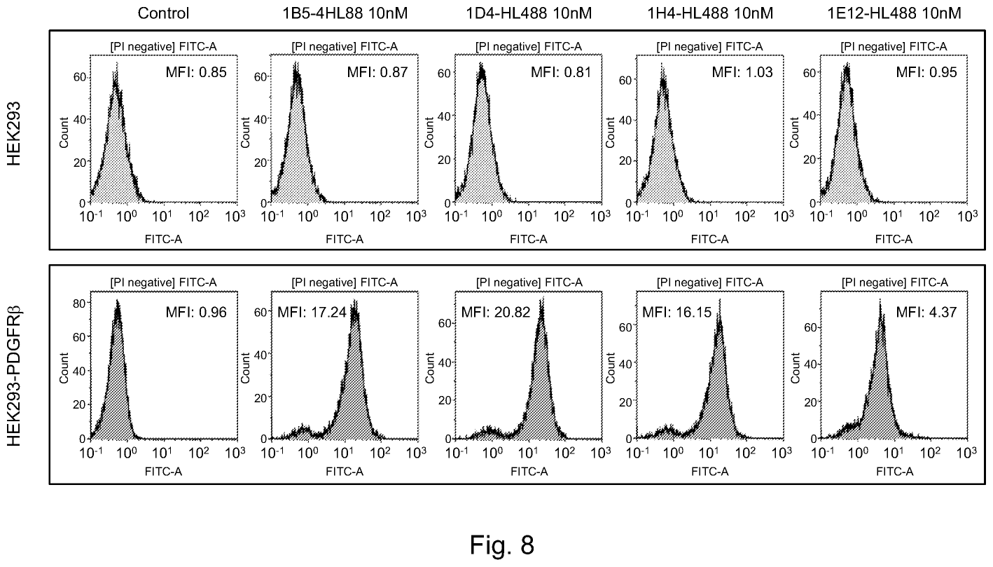

Uptake and binding of VHHs were further analysed by FACS. HEK293 and HEK293-PDGFR β cells were incubated with 10 nM HL488-tagged 1B5, 1D4, 1H4 or 1E12 at 37° C. for 1 h and were measured for fluorescence content. Non-transfected HEK293 showed no VHH uptake/binding. HEK293-PDGFR β, in contrast, took up or bound all tested VHHs. 1B5-HL488, 1D4-HL488 and 1H4-HL488 were effectively taken up/bound by HEK293-PDGFR β, while 1E12-HL488 less efficiently so (see FIG. 8).

These data indicate that VHHs 1B5-HL488, 1D4-HL488, 1H4-HL488 and 1E12-HL488 are bound by or were taken up by cells in a PDGFR β-dependent manner. 1B5-HL488, 1D4-HL488, 1H4-HL488 seem to recognize PDGFBR more efficiently than 1E12-HL488.

### Dose-Response of VHH-HL488 in HEK293-PDGFR β Cells

Next, a VHH detection range was assessed in HEK293 and HEK293-PDGFR β cells.

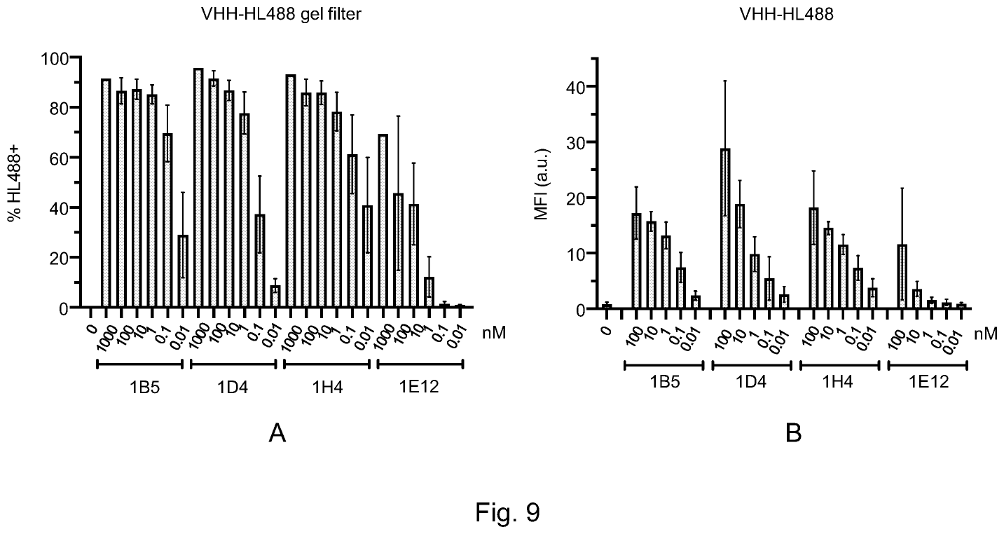

All four VHHs bound and/or were taken up by PDGFR β-expressing cells. HL488-conjugated 1B5, 1D4 and 1H4 were detectable at up to 0.1 nM when looking at either percentage of HL488-positive cells or mean fluorescence intensity of live cells. 1E12-HL488 was detectably bound or taken up at higher concentrations; at 10 nM when looking at percentage of HL488-positive population of at 100 nM when looking at MFI. See FIG. 9.

These findings indicated that the detection limit for 1B5-HL488, 1D4-HL488 and 1H4-HL488 was 0.1 nM, and for 1E12-HL488 100 nM.

### VHH-HL488 Uptake vs Binding, Analysed by FACS

To distinguish cellular binding from internalization, HEK-PDGFR β cells were treated with 1-10 nM VHH-HL488 either at 37° C. (binding and uptake) or at 4° C. (binding) for 1 h and analysed by FACS.

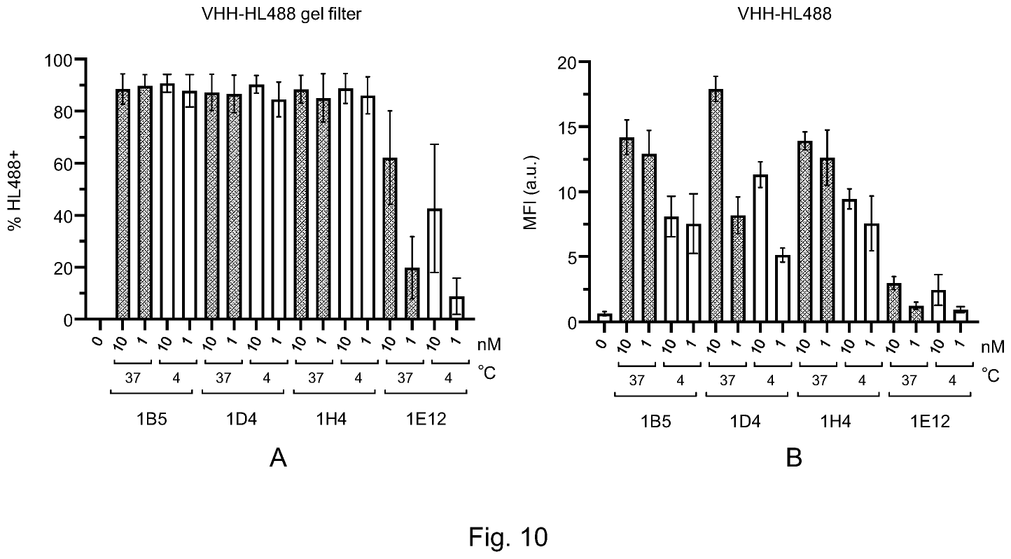

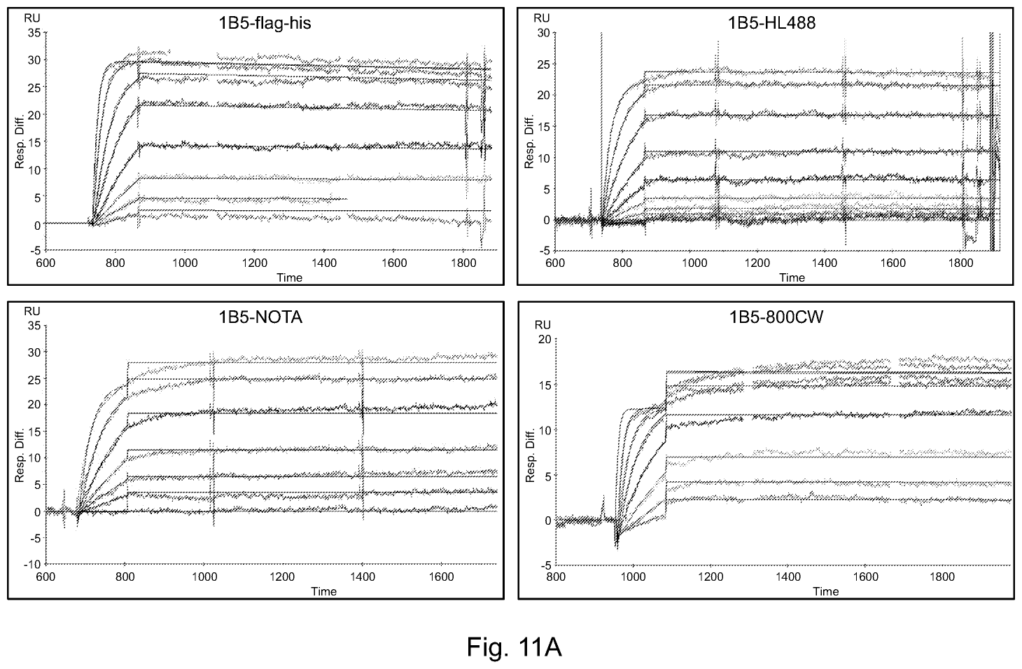

Percentages of HL488-positive cells did not change in response to cold treatment. However, MFI revealed that 1B5-HL488 signal at 4° C. was approximately 52-58% of that at 37° C. The contribution of binding of 1D4-HL488 was 53-61% of total signal and 1H4-HL488 65-66% was binding. See FIG. 10. Hence, the majority of the total HL488 signal appeared to result from VHH cell surface binding.

Conjugated VHHs that were dialysis-purified were also tested. Dialyzed VHH-HL488 conjugates were assessed in FACS for the dose response. 1B4 and 1D4 were detectable by FACS at 1 nM and 1H4 at 10 nM (data not shown).

### Example 6: Biacore SPR Analysis of VHHs and Conjugates Thereof

This example describes the analysis of binding parameters of various VHH's using surface plasmon resonance analysis (SPR).

### Materials and Methods

VHHs 1B5-Flag-His, 1B5-NOTA, 1B5-800CW, 1B5-HL800, 1D4-Flag-His, 1D4-NOTA, 1D4-800CW, 1D4-HL488, 1H4-Flag-His, 1H4-NOTA, 1H4-800CW, 1H4-HL488 and 1E12-Flag-His were synthetized as described herein above.

PDGFR β extra-cellular domain (ECD) with Fc and His tags was purchased from Sino Biological. Protein A from Staphylococcus aureus was purchased from Sigma (P7837).

### Surface Plasmon Resonance (SPR)

SPR analysis was performed using the Biacore 3000 instrument (GE Healthcare). Protein A was chemically bound to a CM4 sensor chip (GE Healthcare) according to the primary amine procedure to approximately 2100 response units (RU). Fc/His-tagged PDGFR β ECD at 0.4-2.07 ng/ul was bound to protein A at flow rate of 35 ul/min. Run buffer HBS-EP (GE Healthcare; 0.01 M HEPES pH 7.4, 0.15 M NaCl, 3 mM EDTA, 0.005% v/v Surfactant P20) was used for VHHs 1D4, 1H4 and 1E12 conjugates at 50, 25, 12.5, 6.25, 3.13, 1.56, 0.78 and 0.39 nM. For 1B5 conjugates, HBS-EP+0.5 M NaSCN was as run buffer to reduce non-specific binding to a control path without PDGFR β ECD. A flow rate 70 ul/min was used for VHH injections in volumes of 150 ul. Regeneration of the sensor CM4 chip was performed by three successive injections of c. 30 s 10 mM glycine-HCl pH 2, 10 mM glycine-HCl pH 1.5, and 0.5 M NaSCN/10 mM NaOH.

### Data Analysis

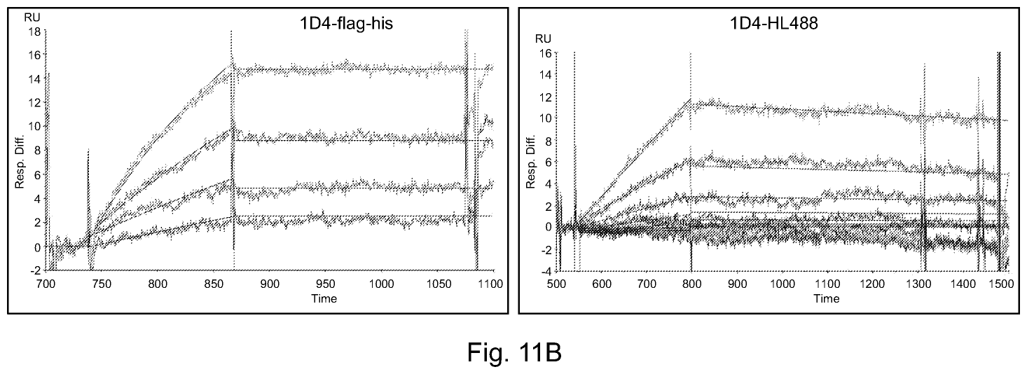

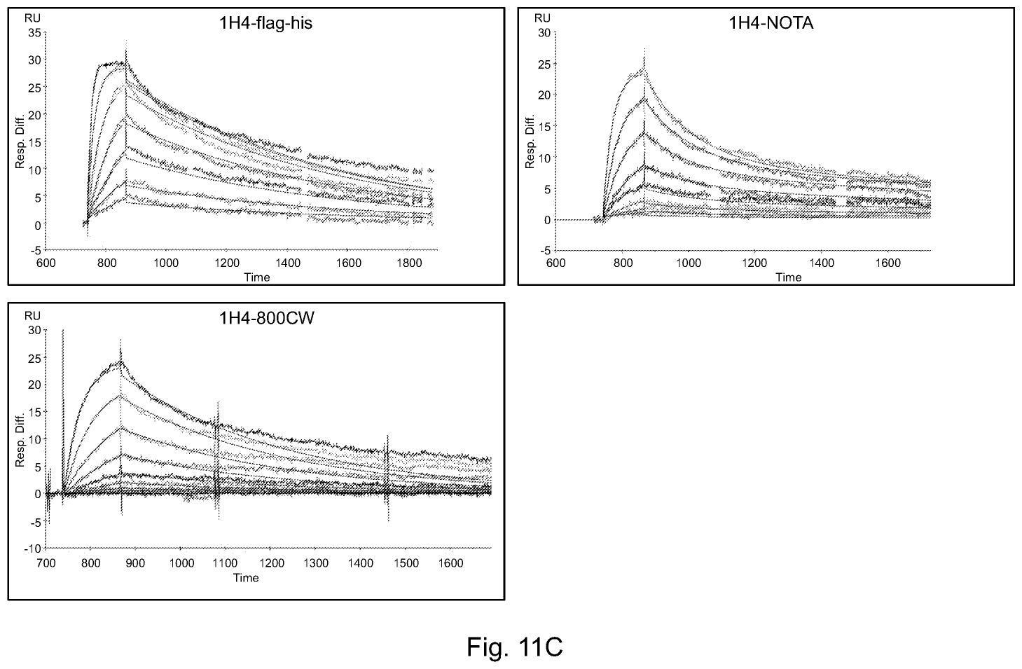

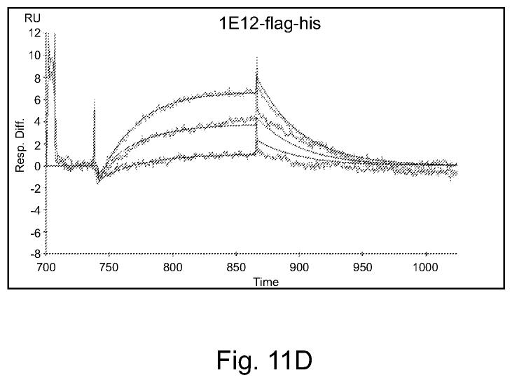

Signal from non-specific binding to the control path was subtracted from the specific signal. Data analysis was performed using the BIA software, using the Lammli 1:1 curve fitting model, unless otherwise stated. Representative results are shown in FIG. 11.

### Example 7: Assessment of pERK and pAKT Activity in Response to VHH Binding

In this example, the potential activation of PDGFR β in response to exemplary VHH's of the invention was investigated. Phosphorylated ERK1 and ERK2 (pERK) or AKT (pAKT) were used as a downstream signaling markers of PDGFR β activity.

### Materials and Methods

### Cells

Human hepatic stellate cells (HHSteC) were bought at SanBio and cultured in Stem Cell medium in 2% FBS, 1% penicillin/streptomycin, 1% growth factors.

### Antibodies

VHH 1B5, 1D4, 1H4 and 1E12, each Flag/His-tagged were produced. Recombinant human TGFB (100-21) and recombinant human PDGF-BB (100-14B) were purchased at Peprotech.

### Western Blot

HHSteC cells were seeded in full growth medium on poly-L-lysine-coated 12-well plates and let adhere until the following day. Cells were washed with PBS and starvation medium was added (0% FBS and growth factors). 24 h after, cells were stimulated with 5 ng/ml TGFb. 24 h post TGFb addition, cells were treated with 50 ng/ml PDGF-BB (approx. 2, 1 nM; positive control) or VHH-Flag/His at 1 uM, 0.1 uM, 0.01 uM for 30 min. Cells were placed on ice and were washed with ice-cold PBS, and lysed directly on wells using SDS sample buffer with 10% mercaptoethanol. Lysates were sonicated and separated on 10% SDS gels and were transferred on a PVDF membrane using standard Western blotting technics. Rabbit pAKT (Ser473), rabbit pERK½ (Thr202/Tyr204) (Cell Signaling) and mouse beta-actin.

### Data Analysis, Statistical Analysis

Band intensities were quantified using Odyssey Image Studio software (LI-COR). pAKT or pERK½ signal was normalised by beta-actin signal that was used as a loading control. Mean band intensity of 1-4 independent experiments with SEM was plotted.

### Results

VHHs 1B4, 1D4, 1H4 and 1E12 were assessed for their potential to activate PDGFR β signaling in fibrotic cells. Human hepatic stellate cells were serum-starved for 24 h and activated with TGFb for 24 h to stimulate fibroblast transformation into myofibroblasts and assumed augmented PDGFR β expression. Cells were treated with human PDGF-BB (50 ng/ml; approx. 2, 1 nM) for 30 min as a positive control for the induction of pAKT and pERK½.

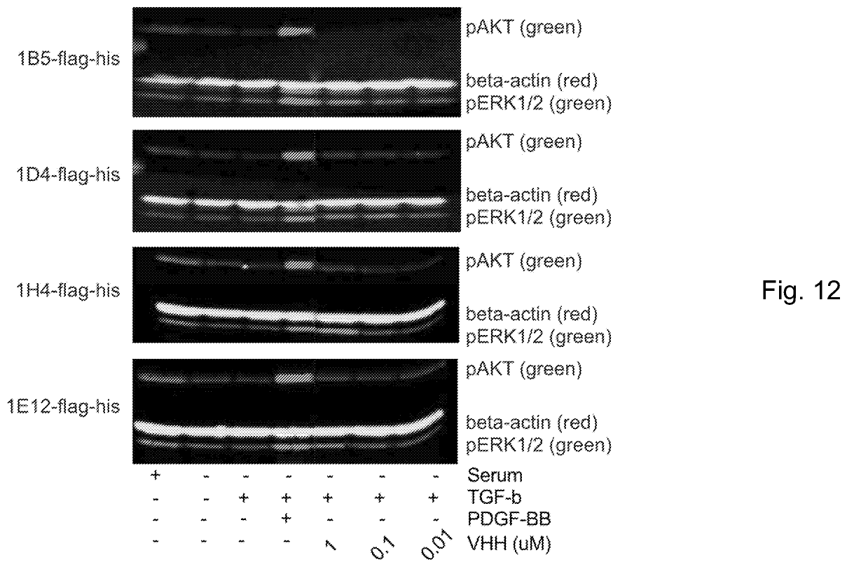

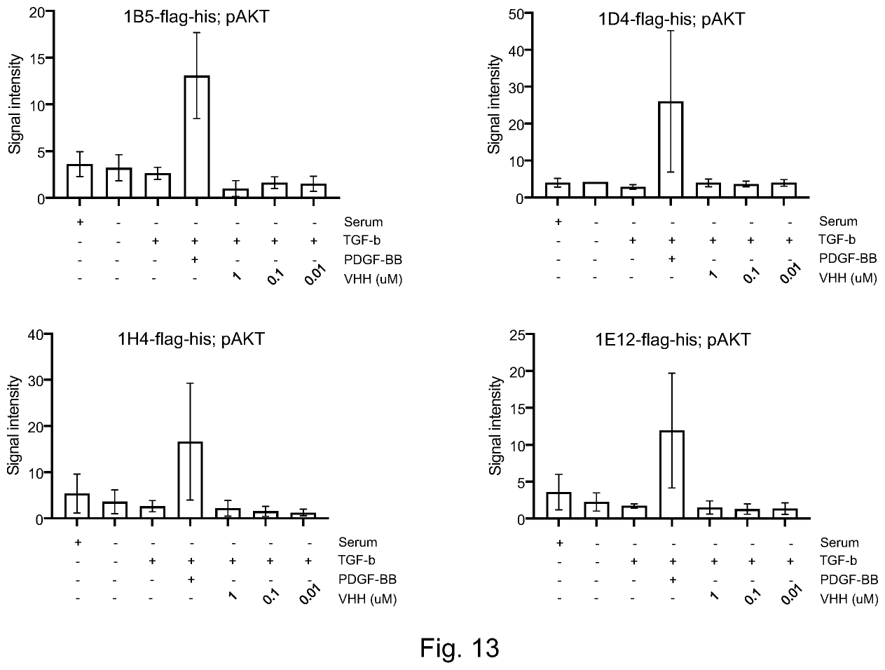

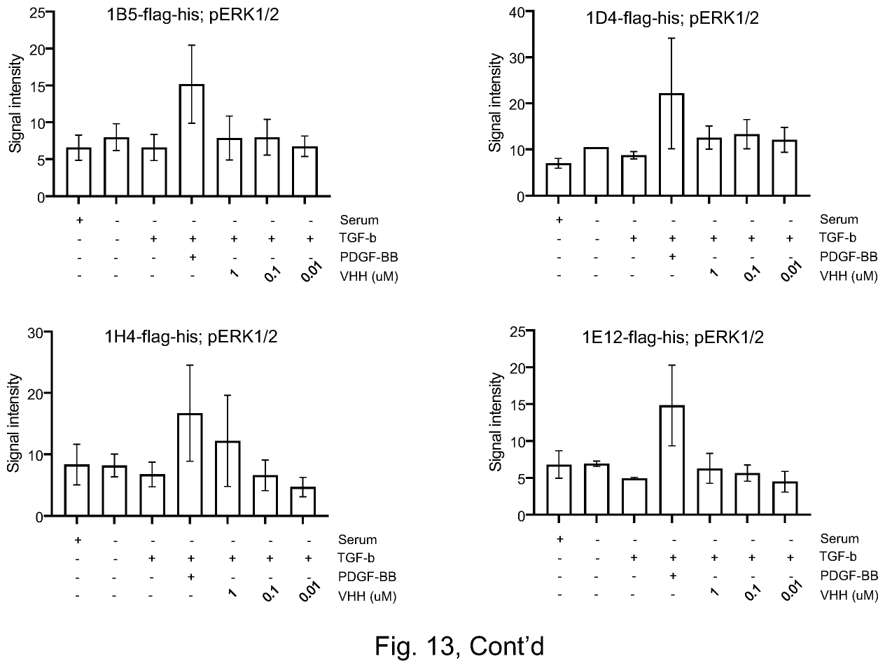

As is shown in FIGS. 12 and 13, incubation of cells with VHHs at any tested concentration had no effect on pAKT, indicating that 1B5, 1D4, 1H4 and 1E12 do not activate the PDGFR β-AKT pathway. pERK½ activity remained at the level of control treatments at 10 or 100 nM. pERK½ was only activated at the highest concentration of 1 μM, which is however an un-physiologically high VHH concentration.

These results suggested that myofibroblasts exposure to VHHs has no effect on (agonistic) PDGFR β signaling via PI3K-AKT. Furthermore, PDGFR β signaling via RAS-RAF-MEK½-ERK½ signaling in myofibroblasts is not affected at VHH concentrations that are considered physiologically relevant.

### Example 8: Receptor Specificity of VHH-Biotin Conjugates

In this example, the receptor specificity of representative VHH's of the invention was investigated for cross-reactivity with the PDGF receptor family member PDGFRa and the epidermal growth factor receptor EGFR.

### Materials and Methods

### Compounds

VHHs 1B5-biotin batch 2 (50uM, received 10.6.2020), 1D4-biotin batch 1 (38.2 uM, received 19.2.2020), 1H4-biotin batch 1 (41.3 uM, 19.2.2020) and 1E12-biotin batch 1 (50.5 uM, received 21.5.2019) were provided by QVQ.

### ELISA

96-well maxisorp (Nunc) immunosorp plates were coated with 2 ug/ml of human PDGFRa (Sino Biological; Cat. Number; 10556-HCCH) or 2 ug/ml human EGFR/HER1/ErbB1 Protein (His Tag, Sino Biological; Cat. Number; 10001-H08H) in PBS, 100 ul per well, at 4C overnight. Wells were washed thrice in PBS. Unspecific binding sites were blocked in 4% BSA/PBS for 1 h at RT, followed by washing thrice in PBS. VHH-biotin conjugates, diluted at 0.01-100 nM in 1% BSA/PBS, were incubated for 1 h at RT, followed by washing thrice in PBS. Streptavidin-horseradish peroxidase (HRP; GeneTex) was diluted 1:40,000 in 1% BSA/PBS and was incubated for 1 h at RT. PDGFRa or EGFR was detected with positive control antibodies rabbit anti-human PDGFRa/CD140a Antibody, (Cat no: 10556-R065) or rabbit anti-human EGFR/HER1/ErbB1 (Catalog no: 10001-R021, both from Sino Biological) diluted in 1% BSA/PBS at 1:5000 and were incubated for 1 h at RT, shaking. Secondary HRP-conjugated anti-rabbit-antibody was diluted at 1:5000 in 1% BSA/PBS and was incubated for 1 h at RT, shaking. Wells were washed 6 times in PBS. o-Phenylenediamine dihydrochloride (OPD; Sigma-Aldrich) was used as HRP substrate at 0.4 mg/ml in 0.05 M phosphate-citrate buffer pH 5.0 with 0.03% sodium perborate (Sigma-Aldrich), prepared according to the manufacturer's recommendations, and was used in volume of 100 ul per well. Reaction was allowed to proceed for 30 min at RT in dark, and was stopped by adding 50 ul of 1.5 M HCl. Optical density was measured at 492 nm on a Synergy H1 microplate reader (BioTek).

### Data Analysis

Average signal from duplicate wells was calculated. Absorbance from non-specific VHH-biotin binding in the absence of PDGFRa or EGFR was subtracted from signal with PDGFRa or EGFR present. Mean absorbance with SEM from 3-6 experiments was graphed on an XY plot using Prism 8 (GraphPad) software. Data was analyzed using nonlinear regression model and equation of log(inhibitor) vs. response, variable slope four parameters. Blanc absorbance value with 0 nM VHH-biotin was included as 10-12.

### Results

### Binding to Human PDGFRα

VHH-biotin binding to human PDGFRa was assessed using an ELISA, where wells were coated with PDGFRa, and VHH-biotin binding was detected using streptavidin-HRP with OPD as a substrate.

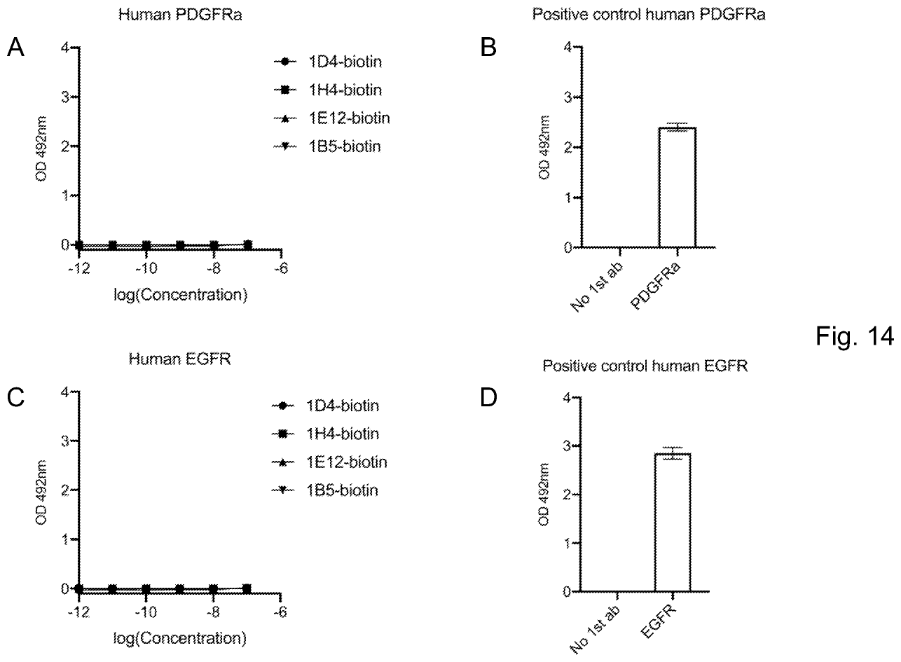

VHH-biotin incubated at 0.01-100 nM showed no affinity to PDGFRa (see FIG. 14A). In contrast, PDGFRa, coated in the wells, was efficiently detected with an anti-PDGFRa antibody and an HRP-conjugated anti-rabbit antibody, demonstrating the presence of functional PDGFRa (FIG. 14B)

### Binding to EGFR

Affinity of biotin-conjugated 1B5, 1D4, 1H4 and 1E12 was assessed on EGFR-coated ELISA assay plates. None of the VHHs, tested at 0.01-100 nM, showed affinity to EGFR (see FIG. 14C). EGFR however was detectable using an anti-EGFR antibody and an HRP-conjugated anti-rabbit antibody, demonstrating the assay function (FIG. 14D).

These data show that none of the VHHs binds to PDGFRa or to EGFR, indicating that VHHs do not cross-react with similar receptors.

### Example 9: Antibody Binding to and Uptake by Human Primary Fibroblasts

Renal fibroblasts were stimulated for myofibroblast transformation by serum stimulation and TGFbeta treatment and were investigated for their ability to take up VHH-HL488. Fluorescent cells were measured on a plate reader. Human hepatic stellate cells were serum starved and TGFbeta-stimulated for myofibroblast transition and were investigated for VHH-HL488 uptake and binding using FACS.

### Materials and Methods

### Cells

Isolated human hepatic stellate cells HHSteC were purchased from ScienCell/Sanbio BV. Cells were cultured in Stellate Cell Medium supplemented with 2% FBS, 1× Stellate Cell Growth Supplement, 100 U/ml penicillin and 100 ug/ml streptomycin, on poly-l-lysine-coated (2 ug/cm²; all reagents from ScienCell) T-75 tissue culture flasks and 12-well plates, using cell culture technics recommended by ScienCell. Renal fibroblasts were obtained from Ruud Bank (UMCG) were grown in DMEM, supplemented with 10% FBS, 1% penicillin/streptocmycin.

### Fibroblast to Myofibroblast Stimulation and Cell Treatment

Renal cells were seeded on 12-well plates and let adhere until the following day. Cells were starved in 0.5% FBS 1% P/S+0.17 mM Ascorbic Acid (VitC) for 18 h. Cells were stimulated with TGFb 5 ng/ml (peprotech 100-21C) for 6 days with daily medium change. On day 6 of stimulation, VHH were added for 24 h. On day 7 post stimulation, VHH were added for 1 h and 0 h, and cells were harvested.

Human hepatic stellate cells were plate of poly-L-lysine coated 12-well plates and were allowed to attach. Cells were starved in 0% FBS, 0% growth factors, starvation medium overnight. Cells were stimulated with 5 ng/ml TGFb for 24 h prior to VHH treatment. VHH were added at 0.1-10 nM for 1 h at 37° C. or at 4° C. prior to harvesting.

### Plate Reader Assay

Medium was gently removed and cells were washed once in PBS. Cells were detached by trypsin, and collected in 10% FBS/PBS. Cells were pelleted by centrifugation and resuspended in 200 ul PBS, and were loaded on black 96-well assay plates. Fluorescence was measured at 488 nm using top optics on Synergy H1 plate reader.

### FACS Assay

Cells were washed and detached by trypsin. Cells were washed twice and resuspended in PFE. Propidium iodine was added at 0,1 ug/ml prior to analyzing cells using the FACS Verse. Dead cells were excluded based on PI content, and live cells were analysed on their HL488 content. Percentage or HL488-positive cells and mean fluorescence intensity of live cells were measured.

### Data Analysis

Mean of 3 independent measurements in renal fibroblast experiments is shown. Mean of two independent experiments of HHSteCs with SEM is shown.

### Results

### VHH Uptake in Renal Fibroblasts

VHH-HL488 uptake in renal fibroblasts and myofibroblasts was assessed. Fibroblasts and myofibrobasts were treated with 10 nM VHH-HL488 for 1 h or 24 h at 37° C., and the resulting cellular fluorescence was analysed on a plate reader. HEK293-PDGFRB cells were used as a positive control.

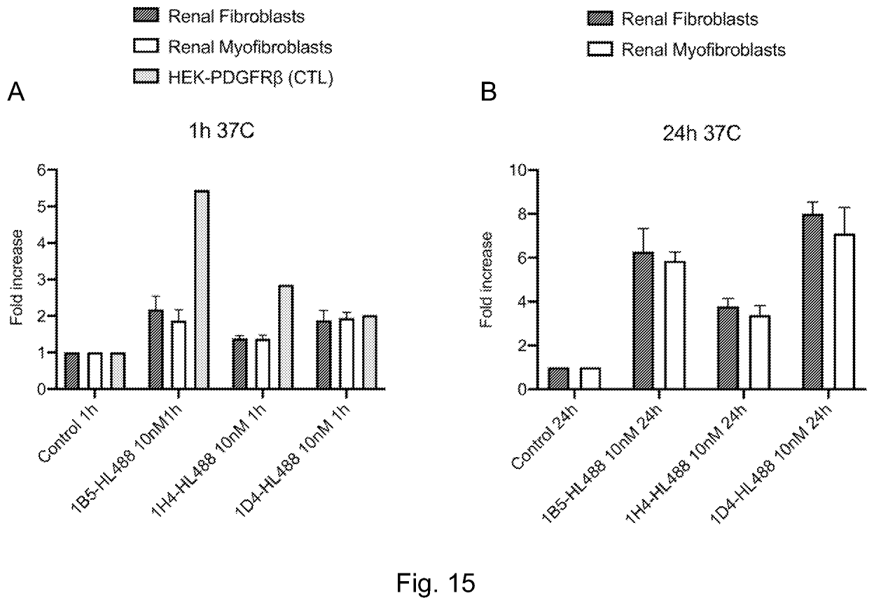

After 1 h incubation, a modest uptake of 1B4 and 1H4 was detectable in both renal fibroblasts and renal myofibroblasts; approximately 1.8-2× increase compared to 0 h control. HEK293-PDGFRB positive control showed uptake of 1B5 and 1H4 uptake after 1 h (FIG. 15A). Uptake of 1B5, 1D4 and 1H4 was more pronounced after 24 h of incubation in both renal fibroblasts and renal myofibroblasts. 1B5 signal was 6× increased, 1H4 was 3-4× increased and 1D4 signal 7-8× increased over the control cells (FIG. 15B). No difference between VHH uptake in renal fibroblasts and myofibroblasts was detected.

### VHH Uptake and Binding in Human Hepatic Stellate Cells

Uptake and binding of VHH-HL488 was assessed on human hepatic stellate cells (HHSteC) using FACS.

First, the effect of serum-starvation and TGFb-stimulation was assessed. Cells grown in full growth medium or starving cells stimulated with TGFb were compared for VHH-HL488 uptake. Serum starvation and TGFb stimulation caused a change the cell morphology, and gating of cell population had to adjusted; hence cells in full growth medium and cells in serum starvation and TGFb-stimulation were compared each to their own 0 nM VHH control. Serum starvation and TGFb stimulation augmented cellular uptake of VHH, compared to cells growing in full growth medium (data not shown). Due to VHH uptake in non-treated cells was very low, the subsequent experiment was conducted only in cells in serum starvation and TGFb stimulation.

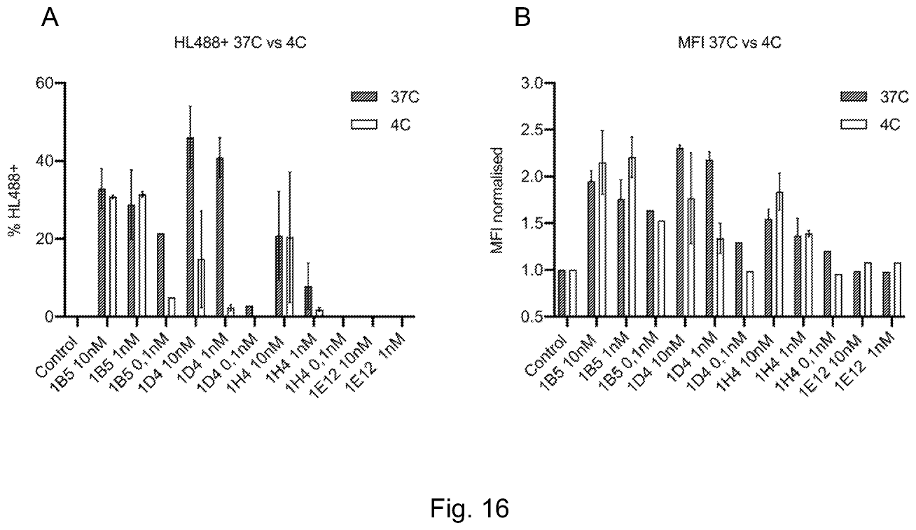

Next, VHH uptake in HHSteC was further assessed. Serum starving TGFb-stimulated HHSteC were treated with 0.1-10 nM VHH-HL488 for 1 h at 37° C. 1B5, 1D4 and 1H4 uptake at 10 nM and 1 nM was detected, however cells did not take up 1E12. See FIG. 16.

Cellular uptake was separated from binding by incubating cells at 4° C. during VHH treatment. Serum starving, TGFb-stimulated HHSteC were incubated with 0.1-10 nM VHH-HL488 for 1 h at 4° C. prior to analyzing cellular fluorescence.

VHH 1B5 binding seemed to contribute the majority of the total cellular fluorescence; incubating cells at 37° C. (uptake and binding) vs 4° C. (binding) did not result to much higher signal. Similarly, 1H4 binding alone seemed to contribute to the majority of the total fluorescence. In contrast, 1D4 incubation at 4° C. largely reduced cellular fluorescence, indicating that cellular uptake took place.

These data indicate that primary human cells take up and bind VHH's of the present invention.

### Example 10: Uptake and Binding of VHH-Conjugated Liposomes by PDGFRβ-Expressing Cells

This example summarizes experiments on uptake and binding of antibody-mediated targeting of liposomal formulations to PDGFRβ-expressing cells.

Disease-targeted liposomes are attractive carriers of therapeutic compounds due to their potential to specifically deliver high drug loads to target cells, while being biodegradable and showing low toxicity. VHH 1B5 is an exemplary PDGFRβ-specific nanobody according to the invention that efficiently recognizes PDGFRβ and is internalized by human PDGFRβ-expressing cells. To demonstrate that 1B5 can act as a PDGFRβ targeting molecule for liposomal drug carriers, a series of fluorescently labeled 1B5-liposomes was generated, alongside with HIV-targeted J3RSc nanobody-liposomes as a negative control. Cellular recognition and internalization of 1B5-liposomes and J3RSc-liposomes were investigated in human embryonic kidney cells HEK293 that were stably transfected with human PDGFRβ (HEK293-PDGFRB). HEK293 cells that do not express PDGFRβ, were used as a negative control. Cellular fluorescence measured by a plate reader was used as a readout of binding and/or uptake.

### Materials and Methods

### Compounds

Liposomes were composed of dipalmitoyl phosphatidylcholine, cholesterol and poly(ethylene glycol)-distearoyl phosphatidylethanolamine, and contained 20 mM calcein and 0.1 mol % rhodamine phosphatidylethanolamine (PE).

Liposome conjugates (batch 12.3.2020, all 10 mM of lipid) were obtained using a conventional procedure. In brief, the selected nanobodies were transferred via a post-insertion technique, wherein micelles comprising maleimide-PEG-DSPE and PEG-DSPE were incubated with the liposomes at elevated temperature (Allen™, et al, Use of the post-insertion method for the formation of ligand-coupled liposomes, Cellular & Molecular Biology Letters 2002, 7(3):889-94). VHH 1B5 or VHH J3RSc (from QVQ) was conjugated to liposomes at ratios of 0, 1, 3, 10, 30 or 100 nanobodies/liposome. Non-targeted liposomes without nanobodies served as a negative control.

### Cell Lines

HEK293 cells were purchased from ECACC/Sigma Aldrich and were cultured in DMEM high glucose supplemented with 10% FCS and 1% penicillin/streptomycin. HEK293 cells stably expressing PDGFRβ (HEK293-PDGFRβ) were constructed by and obtained from Zealand Pharma. The cell line was created using the iDimerize™ Inducible Heterodimer System (catalogue number 635067, Takara Bio). HEK293-PDGFRβ were cultured in Dubecco's Modified Eagle Medium (DMEM) high glucose, supplemented with 10% FBS, 1% sodium pyruvate, 1% non-essential amino acids, 1% L-glutamine, 1% penicillin/streptomycin and 300 ug/ml hygromycin.

### Plate Reader

Cells growing in a culture flask were trypsinised and counted. Cells were resuspended in full growth culture medium at density of 2 million cells/ml and were aliquoted in Eppendorf tubes containing 200.000 cells in volumes of 100 ul. Where cold incubations were used, cells were incubated on ice for 20 min to reach the target temperature prior to adding VHHs. Liposome-VHH conjugates were vortexed, added into cell suspension and gently mixed gently. Cells were incubated at 37° C. or on ice for indicated times until washing three times in 1 ml of cold PBS. Cell pellets was resuspended in PBS in final volumes of 200 ul and were loaded on black 96-well plates for fluorescent measurement at 485/528 nm (calcein) and 560/601 nm (rhodamine PE) on a Synergy H1 microplate reader (BioTek), using top optics with gain of 100.

### Data Analysis, Statistical Analysis

Non-targeted control liposome without VHH (liposome-0) was used as a reference sample to normalize raw fluorescence values. Mean normalized fluorescence from multiple independent experiments is shown with standard error of the mean (SEM). Number of replications are indicated in figure legends.

Data was analysed using Prism 8 software (GraphPad) and 2-way ANOVA and Dunnett's multiple comparison test where non-targeted liposome 0 served as a control sample. Legends; ns. statistically not significant; P≥0.05, *; P=0.01-0.05, **; P=0.001-0.01, ***; P=0.0001-0.001, ****; P<0.0001.

### Results

### 1B5-Liposomes are Taken up by HEK293-PDGFRβ Cells

PDGFRβ-targeted liposomes with different nanobody-to-liposome ratios, ranging from 1, 3, 10, 30 and 100, were generated. J3RSC is a VHH nanobody that recognizes HIV and has no epitope in human cells, and was used to generate J3RSc-liposomes that served as negative controls. Liposomes were loaded with calcein, which is self-quenched at high concentrations in liposomal preparations, and liposomal release and dilution of calcein into cytosol results to increased fluorescence. The liposomal bilayer was labeled with rhodamine PE, which renders plasma membrane fluorescent upon membrane fusion.

Incubation of HEK293-PDGFRβ cells with 500 uM 1B5-liposome-100 at 37° C., the temperature which allows cells to both bind and take up compounds, resulted in a 3.4-fold increased calcein florescence. Incubation of HEK293-PDGFRβ on ice, which blocks active uptake processes, lead to a lower in calcein signal, 1.8-fold over non-targeted control. Rhodamine PE signal of 1B5-liposome-100 increased by 2.4-fold in HEK293-PDGFRβ at 37° C., and incubation of ice reduced the signal (data not shown). These findings indicated that 1B5-liposome-100 was both bound and taken up by cells. The internalization was specific and PDGFRβ-dependent, since HEK293 took up no liposome constructs, and the HIV-targeted J3RSc-liposome-100 was not taken up by either cell line (data not shown).

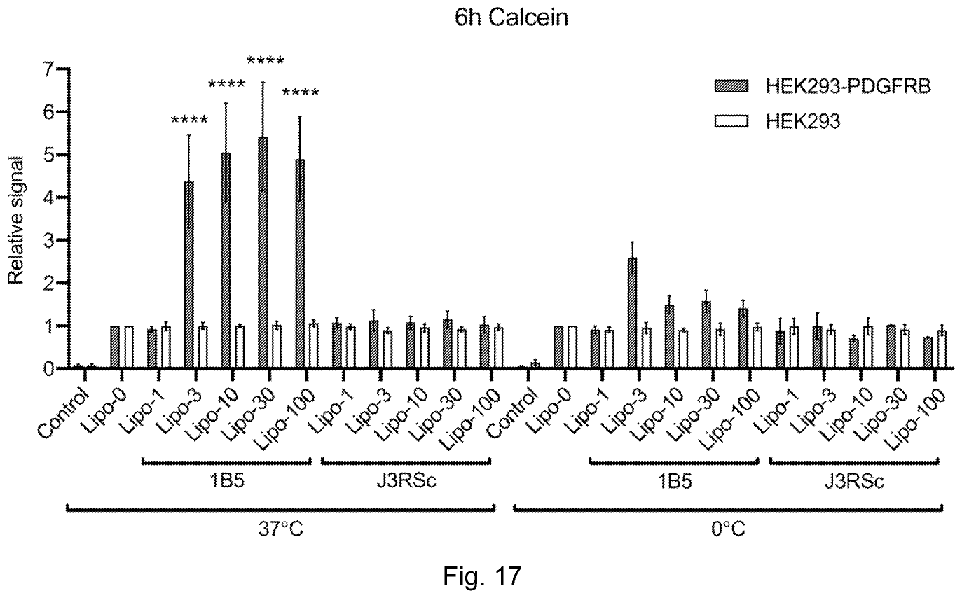

To investigate which ratio of nanobody to liposome conjugates was the most efficient in terms of cellular uptake, 1B5 nanobody to liposome constructs at ratios 1, 3, 10, 30 and 100 were tested. HEK293-PDGFRβ and HEK293 cells were incubated with 500 uM liposome constructs for 6 h at 37° C. or on ice. Calcein fluorescence showed that 1B5-liposome conjugates with ratios of 3, 10, 30 and 100 were all efficiently taken up at 37° C. by HEK293-PDGFRβ but not by HEK293, and that signal was largely inhibited after incubation on ice (FIG. 17). Rhodamine signal similarly showed efficient 1B5-liposome uptake at nanobody/liposome ratios of 3, 10, 30 and 100 (data not shown). Incubation on ice prevented uptake of 1B5-liposome 10, 30 and 100, but not that of liposome at ratio 3. J3RSc-liposome constructs did not bind or were not taken up by either HEK293-PDGFRβ or HEK293 cells.

These data show that 1B5-liposome constructs are taken up by active processes by cells, and that the uptake and binding occur via PDGFRβ. Conjugate proportions of 3, 10, 30 and 100 nanobodies per liposomes are suitable ratios, while 1 nanobody per liposome is less sufficient for binding or uptake.

### Uptake of Liposome-1B5 is Time-Dependent

Next, a time course experiment of 1B5-liposome uptake was performed. HEK293-PDGFRβ cells were incubated with 1B5-liposome-100 or with the negative controls non-targeted liposome-0 or HIV-targeted J3RSc-liposome-100 for 0 h, 1 h, 3 h or 6 h. Background calcein fluorescence from non-targeted liposome-0 control was found increasing over time, hence 1B5-liposome and J3RSc-liposome values of each time point were normalized by its own liposome-0 control. Non-specific rhodamine fluorescence did not change over time (data not shown)

HEK293-PDGFRβ showed time-dependent 1B5-liposome uptake. After 0 h and 1 h of incubation, 2.2 and 2.8-fold 1B5-liposome-100 binding and/or uptake was observed based on calcein fluorescence, however this was not statistically significant. After 3 h and 6 h, 4.7 and 5.1-fold calcein-based 1B5-liposome uptake was detected. Rhodamine PE-based detection of 1B5-liposome uptake was found significant after 6 h of incubation (FIG. 3). Consistent to findings in FIG. 17, these data demonstrate that 1B5-liposome-100 uptake occurred specifically via PDGFRβ, as HEK293 cells that lack PDGFRβ showed no uptake, and HIV-targeted J3RSc-liposome-100 did not bind either cell line. In agreement with timing of active cellular transport such as endocytosis, longer incubation time yielded higher 1B5-liposome uptake.

### Dose-Response Assays

Next, dose-response experiments were performed to find the minimum effective dose of 1B5-liposome constructs. HEK293-PDGFRβ and HEK293 cells were treated with 500, 250, 125, 62.5. 31.3, 15.6, 7.8u, 3.9, 2.0, 1.0 or 0.5 uM non-targeted liposome-0, 1B5-liposome-100 or J3RSc-liposome-100 for 4 h at 37° C.

Liposome-VHH dilutions of 125, 62.5, 31.3, 15.7 and 7.8 uM exhibited the widest window between non-specific liposome-0 or J3RSc-liposome-100, and specific 1B5-liposome-100 calcein uptake), with 31.3 uM showing the highest signal. Rhodamine PE signal indicated that liposome dilutions at 250, 125, 62.5 and 31.3 uM showed almost equal differences between control and 1B5-liposome-100 uptake (data not shown).

Hence, liposome-1B5 at concentrations of 31.3-125 uM of liposome are very effective in HEK293-PDGFRβ uptake experiments.

### Conclusion

PDGFRβ-targeted liposomes specifically bind PDGFRβ and are taken up by HEK293-PDGFRβ cells using active transport mechanisms. VHH-liposome conjugations at ratio of 3, 10, 30 and 100 nanobodies per liposome are efficiently taken up via PDGFRβ, while the conjugate with 1 nanobody per liposome does not associate with PDGFRβ expressing cells. VHH-liposome uptake is time-dependent and occurs efficiently at 31.3-125 uM liposomal concentrations.

### Example 11: Stability of Conjugated Non-Agonistic PDGFRβ Antibodies

In this example, the stability of conjugated VHH's according to the invention was investigated in vitro and ex vivo.

### Freeze-Thawing Experiments

Stability of biotin conjugates of VHHs 1B5, 1D4, 1H4 and 1E12 after 3 freeze-thaw cycles were investigated, assessed by their binding capacity to human PDGFRβ.

Frozen aliquots underwent freeze-thaw cycles for 3× (=4×frozen, 4×thaw). To perform a freeze-thaw cycle, an aliquot was taken from −20° C. and placed on a benchtop for one hour at RT, then returned to −20° C. for at least until the following day. Boiled sample was heated at 80° C. for 8 h in a heat block, then the heat block was turned off and let cool overnight, with sample on it. Boiled sample was stored frozen the following day.

VHH integrity was assessed based on retained ability to bind to human PDGFRβ extracellular domain, and bound VHH were detected via streptavidin-HRP or via anti-VHH antibody and an HRP-conjugated anti-rabbit antibody. Freeze-thawing appeared to have no effect on VHHs 1B5 and 1D4. VHHs 1H4 and 1E12 seemed to bind PDGFRβ with a slightly reduced affinity.

### In Vivo Stability

This example summarizes measurements of VHH-800CW conjugate concentration in mouse plasma and blood cells. Nanobody conjugates 1B5-800CW, 1D45-800CW, 1H4-800CW and 1E12-800CW were dissolved in PBS at concentration of 400 ug/ml, and administered at 40 ug in volume of 100 ul per mouse. Adult male C57BI/6 mice (n=9, weight approx. 25-30 g) were injected once via i.v. route. Blood was drawn from cheek vein at time points 5 min, 20 min, and 60 min, 3 time points per animal.

Measurements were performed using direct fluorescence measurement in plasma or ELISA. Direct fluorescence measurement indicated that 1B5-800CW half-life in plasma was 5.675 min, 1D4-800CW half-life in plasma is 4.223 min, 1H4-800CW 5.106 min, and 1E12-800CW 4.828 min. ELISA-based detection indicated that 1B5-800CW half-life was 3.59 min, 1D4-800CW 3.89 min, 1H4-800CW 4.318 min, and 1E12-800CW 6.064 min.

### Example 12: Ex Vivo Near Infrared Imaging of Conjugated VHHs

This example describes the ex vivo near infrared imaging of mouse specific VHH's in mice with bleomycin induced pulmonary fibrosis.

### Materials and Methods

Anti-PGRFRβ VHH 1E12-800CW was synthesized as described herein above. Negative control anti-HIV VHH J3RSc-800CW was obtained from QVQ, Utrecht, The Netherlands.

Male C57BL/J6 mice (10-12 weeks) received a single dose of Bleomycin intratracheally (0.08 mg/kg in 50 uL PBS) to induce pulmonary fibrosis. Control mice received an equal volume of vehicle alone. Three weeks after the start of the bleomycin application, mice were injected with 40 μg VHH-conjugate in 40 μL PBS and whole animals were scanned 2-6 hours after probe injection using fluorescence mediated tomography (IVIS, Perkin Elmer). Immediately after the last in vivo scan, animals were euthanized and the lungs were excised and scanned ex vivo in the IVIS.

### Results

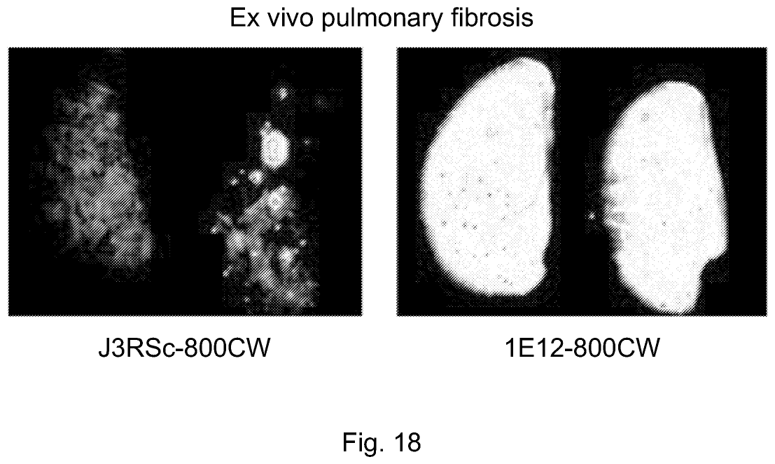

As shown in FIG. 18, VHH 1E12-800CW accumulates specifically in the fibrotic lungs as is reflected by the colour intensity in the tissue that contains cells expressing PDGFβ receptors. In contrast, negative control VHH J3RSc that only binds to HIV receptors, does not exhibit any accumulation in fibrotic tissue.

1. An antibody that specifically binds to the Platelet-derived growth factor receptor beta (PDGFR β) with a binding affinity of less than 10 nM.

2. The PDGFR β antibody according to claim 1, which binds to the dimeric form of the PDGFR β with a binding affinity of less than 10 nM.

3. The PDGFR β antibody according to claim 1, which binds to human PDGFR β with a binding affinity of less than 10 nM.

4. The PDGFR antibody according to claim 1, which is a single heavy chain variable domain (VHH) antibody.

5. A PDGFR β antibody according to claim 1, comprising
a heavy chain CDR1, CDR2 and CDR3 sequence as defined by ID NO: 1, 5 and 9, respectively, or sequences showing at least 90% identity thereto;
heavy chain CDR1, CDR2 and CDR3 sequence as defined by ID NO: 2, 6 and 10, respectively, or sequences showing at least 90% identity thereto;
a heavy chain CDR1, CDR2 and CDR3 sequence as defined by ID NO: 3, 7 and 11, respectively, or sequences showing at least 90% identity thereto; or
a heavy chain CDR1, CDR2 and CDR3 sequence as defined by ID NO: 4, 8 and 12, respectively, or sequences showing at least 90% identity thereto.

6. The PDGFR β antibody of claim 5, comprising a heavy chain variable region comprising a sequence as set forth in SEQ ID NO: 25-32, or a variant or a derivative thereof.

7. The PDGFR β antibody according to claim 6, comprising a heavy chain variable region comprising a sequence as set forth in SEQ ID NO: 25 or 26.

8. The PDGFR β antibody of claim 1, comprising a peptide-tag allowing for purification, a tag allowing for site-specific antibody conjugation, and/or a tag allowing for targeting and/or retention in an organ of interest.

9. The PDGFR β antibody according to claim 1, further comprising a detectable label, a (radio)therapeutic agent, a carrier, or any combination thereof.

10. The PDGFR β antibody according to claim 9, wherein the detectable label is an in vivo detectable label which can be detected in vivo using nuclear magnetic resonance NMR imaging, near-infrared imaging, positron emission tomography (PET), scintigraphic imaging, ultrasound, or fluorescence analysis.

11. The PDGFR β antibody according to claim 9, wherein the therapeutic agent is selected from the group consisting of a radionuclide, a cytotoxin, and a chemotherapeutic agent.

12. The PDGFR β antibody according to claim 9, wherein the carrier is selected from the group consisting of a liposome, a polymersome, a nanoparticle and a microcapsule.

13. A bivalent bispecific, bivalent biparatopic or multispecific binding compound comprising a PDGFR β antibody according to claim 1.

14. A nucleic acid encoding an antibody of claim 1.

15. A method for producing the antibody of claim 1, the method comprising expressing the nucleic acid in a relevant host cell and recovering the thus produced antibody from the cell, optionally further comprising providing the antibody with a detectable label, a therapeutic agent, a carrier, or any combination thereof.

16. A therapeutic composition, a diagnostic composition, or a combination thereof, comprising one or more antibodies according to claim 1.

17. The PDGFR β antibody according to claim 1, for use as targeting agent, diagnostic agent, therapeutic agent or any combination thereof.

18. The PDGFR β antibody of claim 1 for use in a method of diagnosing and/or treatment of a PDGF-mediated disease or medical condition in a mammal.

19. The PDGFR β antibody for use according to claim 18, wherein said PDGF-mediated disease or medical condition is cancer, restenosis, fibrosis, angiogenesis, renal disease or cardiovascular disease.

20. The PDGFR β antibody for use according to claim 18, wherein the method for treatment further comprises the administration of at least one further (chemo/(radio)therapeutic agent.

21. A method for diagnosing and/or treating of a PDGF-mediated disease or medical condition in a mammalian subject comprising administering to the subject a PDGFR β antibody of claim 1.

22. The method according to claim 21, wherein said PDGF-mediated disease or medical condition is cancer, restenosis, fibrosis, angiogenesis, pulmonary disease, renal disease or cardiovascular disease.

23. The method of treatment according to claim 21, wherein the treatment further comprises the administration of at least one further (chemo)therapeutic agent.


SEQUENCE LISTING (40 sequences)

SEQ ID NO: 1 - protein, 5 aa, Artificial Sequence
  CDR1 1B5
  MISC_FEATURE (1)..(1): X is E or T
XSAMS

SEQ ID NO: 2 - protein, 5 aa, Artificial Sequence
  CDR1 1H4
PFAMA

SEQ ID NO: 3 - protein, 5 aa, Artificial Sequence
  CDR1 1D4
RDVVG

SEQ ID NO: 4 - protein, 5 aa, Artificial Sequence
  CDR1 1E12
  MISC_FEATURE (1)..(1): X is A or P
  MISC_FEATURE (4)..(4): X is M or T
  MISC_FEATURE (5)..(5): X is G or S
XYLXX

SEQ ID NO: 5 - protein, 11 aa, Artificial Sequence
  CDR2 1B5
GISSGGGRSTT

SEQ ID NO: 6 - protein, 10 aa, Artificial Sequence
  CDR2 1H4
RISWTAGRTE

SEQ ID NO: 7 - protein, 9 aa, Artificial Sequence
  CDR2 1D4
LVTRSYSTY

SEQ ID NO: 8 - protein, 10 aa, Artificial Sequence
  CDR2 1E12
  MISC_FEATURE (5)..(5): X is P or A
  MISC_FEATURE (8)..(8): X is T or S
AWRWXTSXTY

SEQ ID NO: 9 - protein, 13 aa, Artificial Sequence
  CDR3 1B5
TGYCSGYNCNFAP

SEQ ID NO: 10 - protein, 13 aa, Artificial Sequence
  CDR3 1H4
RFVPSSGPTVFDT

SEQ ID NO: 11 - protein, 18 aa, Artificial Sequence
  CDR3 1D4
DQGFCSGSGCYDWRTFHV

SEQ ID NO: 12 - protein, 17 aa, Artificial Sequence
  CDR3 1E12
  MISC_FEATURE (3)..(3): X is G or E
  MISC_FEATURE (9)..(9): X is A or P
  MISC_FEATURE (12)..(12): X is P or E
  MISC_FEATURE (14)..(14): X is A or P
RRXILYGPXTSXTXYDF

SEQ ID NO: 13 - protein, 5 aa, Artificial Sequence
  CDR1 1B5
ESAMS

SEQ ID NO: 14 - protein, 5 aa, Artificial Sequence
  CDR1 1H4
PFAMA

SEQ ID NO: 15 - protein, 5 aa, Artificial Sequence
  CDR1 1D4
RDVVG

SEQ ID NO: 16 - protein, 5 aa, Artificial Sequence
  CDR1 1E12
AYLMG

SEQ ID NO: 17 - protein, 11 aa, Artificial Sequence
  CDR2 1B5
GISSGGGRSTT

SEQ ID NO: 18 - protein, 10 aa, Artificial Sequence
  CDR2 1H4
RISWTAGRTE

SEQ ID NO: 19 - protein, 9 aa, Artificial Sequence
  CDR2 1D4
LVTRSYSTY

SEQ ID NO: 20 - protein, 10 aa, Artificial Sequence
  CDR1 1E12
AWRWPTSTTY

SEQ ID NO: 21 - protein, 13 aa, Artificial Sequence
  CDR3 1B5
TGYCSGYNCNFAP

SEQ ID NO: 22 - protein, 13 aa, Artificial Sequence
  CDR3 1H4
RFVPSSGPTVFDT

SEQ ID NO: 23 - protein, 18 aa, Artificial Sequence
  CDR3 1D4
DQGFCSGSGCYDWRTFHV

SEQ ID NO: 24 - protein, 17 aa, Artificial Sequence
  CDR3 1E12
RRGILYGPATSPTAYDF

SEQ ID NO: 25 - protein, 123 aa, Artificial Sequence
  1B5 HC
EVQLVESGGGLVQPGGSLRLSCVASGFSFSESAMSWVRQAPGKGLEWLSGISSGGGRSTTYAESVEGRFTISRDDAKNTLMLQMNSLKSEDTAVYYCAKTGYCSGYNCNFAPRGQGTQVTVSS

SEQ ID NO: 26 - protein, 123 aa, Artificial Sequence
  1B5* HC
DVQLVESGGGLVQPGGSLRLSCVASGFSFSESAMSWVRQAPGKGLEWLSGISSGGGRSTTYAESVEGRFTISRDDAKNTLMLQMNSLKSEDTAVYYCAKTGYCSGYNCNFAPRGQGTQVTVSS

SEQ ID NO: 27 - protein, 126 aa, Artificial Sequence
  1D4 HC
EVQLVESGGGLVQAGDSLRLSCAVSGRAQSRDVVGWFRQAPGKEREFLALVTRSYSTYYGASVKGRFTISRDNAKNTVHLEMNSLKPEDTGVYYCAADQGFCSGSGCYDWRTFHVWGQGTQVTVSS

SEQ ID NO: 28 - protein, 126 aa, Artificial Sequence
  1D4* HC
DVQLVESGGGLVQAGDSLRLSCAVSGRAQSRDVVGWFRQAPGKEREFLALVTRSYSTYYGASVKGRFTISRDNAKNTVHLEMNSLKPEDTGVYYCAADQGFCSGSGCYDWRTFHVWGQGTQVTVSS

SEQ ID NO: 29 - protein, 122 aa, Artificial Sequence
  1H4 HC
EVQLVESGGGLVQPGGSLRVSCSASWRTGNPFAMAWFRQAPGKERELIARISWTAGRTEYAESAKGRFTISRDVAKKTMYLQMNSLTPEDTGVYFCAARFVPSSGPTVFDTWGQGTQVTVSS

SEQ ID NO: 30 - protein, 122 aa, Artificial Sequence
  1H4* HC
DVQLVESGGGLVQPGGSLRVSCSASWRTGNPFAMAWFRQAPGKERELIARISWTAGRTEYAESAKGRFTISRDVAKKTMYLQMNSLTPEDTGVYFCAARFVPSSGPTVFDTWGQGTQVTVSS

SEQ ID NO: 31 - protein, 126 aa, Artificial Sequence
  1E12 HC
EVQLVESGGGLVQAGGSLRLSCAPSERSFSAYLMGWFRQAQGKEREFVAAWRWPTSTTYYSDSGKGRFTITGDNAKNTVELEMNSLKPEDTAVYYCAARRGILYGPATSPTAYDFWGQGTQVTVSS

SEQ ID NO: 32 - protein, 126 aa, Artificial Sequence
  1E12* HC
DVQLVESGGGLVQAGGSLRLSCAPSERSFSAYLMGWFRQAQGKEREFVAAWRWPTSTTYYSDSGKGRFTITGDNAKNTVELEMNSLKPEDTAVYYCAARRGILYGPATSPTAYDFWGQGTQVTVSS

SEQ ID NO: 33 - protein, 9 aa, Artificial Sequence
  amino acid linker
GGGGSGGGS

SEQ ID NO: 34 - protein, 5 aa, Artificial Sequence
  Germline family
KGLEW

SEQ ID NO: 35 - protein, 5 aa, Artificial Sequence
  germline family
KEREL

SEQ ID NO: 36 - protein, 5 aa, Artificial Sequence
  germline family
KEREF

SEQ ID NO: 37 - protein, 123 aa, Artificial Sequence; identical to SEQ ID NO: 26
  QPD1B5

SEQ ID NO: 38 - protein, 122 aa, Artificial Sequence; identical to SEQ ID NO: 30
  QPD1H4

SEQ ID NO: 39 - protein, 126 aa, Artificial Sequence; identical to SEQ ID NO: 28
  QPD1D4

SEQ ID NO: 40 - protein, 126 aa, Artificial Sequence; identical to SEQ ID NO: 32
  QPD1E12